(19) United States
(12) Patent Application Publication
(10) Pub. No.: US 2006/0217710 A1
(43) Pub. Date: Sep. 28, 2006

(54) Occipital fixation system and method of use

(76) Inventor: M. Samy Abdou, San Diego, CA (US)

Correspondence Address: FISH & RICHARDSON, PC, P.O. BOX 1022, MINNEAPOLIS, MN, 55440-1022, US

(21) Appl. No.: 11/371,177

(22) Filed: Mar. 7, 2006

Related U.S. Application Data
(63) Continuation-in-part of application No. 11/153,258, filed on Jun. 14, 2005.
(60) Provisional application No. 60/659,675, filed on Mar. 7, 2005.
(60) Provisional application No. 60/718,509, filed on Sep. 19, 2005.
(60) Provisional application No. 60/731,888, filed on Oct. 31, 2005.

(51) Int. Cl. A61B 17/00

(52) U.S. Cl. 606/54

(57) ABSTRACT

Disclosed are skull fixation assemblies and corresponding components. The assemblies include a multi-axial occipito-cervical connection system that enables elongate interconnectors, such as rods, to be coupled in a manner that permits relative movement in one or more planes. The system includes a locking mechanism that can be actuated to lock the relative positions of the elongate interconnectors.

### REFERENCE TO PRIORITY DOCUMENT

This application claims priority of the following co-pending U.S. Provisional Patent Applications: (1) U.S. Provisional Patent Application Ser. No. 60/659,675, filed Mar. 7, 2005; (2) U.S. Provisional Patent Application Ser. No. 60/718,509, filed Sep. 19, 2005; and (3) U.S. Provisional Patent Application Ser. No. 60/731,888, filed Oct. 31, 2005. Priority of the aforementioned filing dates is hereby claimed, and the disclosures of the Provisional Patent Applications are hereby incorporated by reference in their entirety.

This application is a continuation-in-part of co-pending U.S. patent application Ser. No. 11/153,258 entitled “Occipital Fixation System and Method of Use”, filed Jun. 14, 2005, which is incorporated by reference in its entirety.

### BACKGROUND

The present disclosure relates to bone fixation systems, components thereof, and methods of implant placement. These systems are used to adjust, align and maintain the spatial relationship(s) of adjacent bones or bony fragments after surgical reconstruction of skeletal segments. More specifically, but not exclusively, the present disclosure relates to devices that fixate the skull onto the cervical spine.

Whether for degenerative disease, traumatic disruption, infection or neoplastic invasion, surgical reconstruction of the bony skeleton is a common procedure in current medical practice. Regardless of anatomical region or the specifics of the reconstructive procedure, many surgeons employ an implantable bone fixation device to adjust, align and maintain the spatial relationship(s) of adjacent bones or bony fragments during postoperative healing. These devices are usually attached to bone using screws, cables or similar fasteners and act to share the load and support the bone as healing progresses.

The region of articulation between the base of the skull and the upper cervical spine is known as the cranio-vertebral junction. This critical intersection houses and protects the upper aspect of the spinal cord as it emerges from the lower end of the brainstem. Instability of this region can lead to severe spinal cord injury with devastating neurological deficits and death. In order to avoid neurological injury in patients with cranio-vertebral instability, surgical fixation of the hyper-mobile region is performed. Unfortunately, this procedure is technically demanding and the shortcomings of available fixation devices add to the challenges of surgical stabilization.

### SUMMARY

The shortcomings of occipito-cervical fixation devices include:

a) The fixation device is usually anchored onto the sub-occipital bone in the midline and/or on either side of midline. While bone screws, cables, hooks, clamps and other fasteners have been used, bone screws are used most commonly. In attachment onto the sub-occipital bone, the screws are placed into the underlying bone at a trajectory that is perpendicular to the bone surface. Unfortunately, this trajectory is in line with the rotational forces acting upon the cranium and screws placed in this way experience maximum load. Further, perpendicular screw placement provides sub-optimal resistance to pull-out since the screws do not capture a wedge of bone as they would with non-perpendicular placement. Lastly, this trajectory is not in line with the surgeon's line of vision—increasing the technical difficulty of screw placement and the likelihood of poor positioning.

b) Extensive contouring is often required so that the fixation device can conform to the complex and tortuous anatomy of this region. The contouring is done at the time of surgery and this step increases the length of the operation. Intra-operative countering of orthopedic devices is imprecise and devices that are shaped in this way are less likely to conform well to the regional anatomy. This is especially true in regions of tortuous anatomy like the cranio-vertebral junction. Lastly, the contouring process will introduce a variety of curves and stress-risers into the device that may weaken key components. These factors will collectively increase the likelihood of device failure.

c) While the fixation device provides immediate immobilization of the unstable segment, long term success of the operative procedure requires fusion of the hyper-mobile bones. Indeed, bony fusion is the primary goal of the stabilization procedure and the operation will ultimately fail unless the unstable bony segments fuse together and coalesce into a stable construct. Unfortunately, current fixation systems often ignore the central core of the stabilization procedure—the bone graft. These systems are often bulky and provide insufficient space for graft placement. Even when space is available, they provide no means for retention of the graft at the desired fusion site. Lastly, current fixation systems disadvantageously shield the graft and the fusion site form stress. Since stress is a potent stimulant of bone formation, any reduction in the stress forces borne by the bone graft will necessarily decrease the likelihood of successful fusion.

In view of the proceeding, it would be desirable to design an improved cranio-vertebral fixation device and placement protocol. An improved device desirably provides superior bone fixation at this critical region while greatly increasing the ease of use and the reliability of the implantation process.

Disclosed is a device that attaches onto the skull. Bone screws are placed through the device and into the underlying bone at an angle that is preferably not perpendicular to the bone surface or at a variety of different angles. When the screws are placed at a non-perpendicular angle to bone, they can be longer than those screws placed at a right angle. It also means that a wedge of bone is interposed between the screw shank and the bone surface and this wedge would have to be dislodged before the screw can be avulsed from the bone. These factors increase screw resistance to pull-out in a manner independent of screw design. Lastly, the non-perpendicular screw trajectory places the screw within the surgeon's line of vision, thereby making the screw placement technically easier and more precise.

An interconnecting member or interconnector, such as a rod or plate, is used to connect the occipital attachment to cervical attachment(s). Whereas current art connects these attachments using a rod or plate that requires contouring, the disclosed device uses a connector that is freely positionable. This connection allows placement of the inter-connecting rods/plates without contouring and permit rapid and precise device implantation without placement of stress risers within the implanted device. Several embodiments of the connector are shown and described below.

The fixation systems described herein provide ease of use, reliable bone fixation, and optimal biomechanical advantage. The systems also maximize the likelihood of proper device placement and expedite the operative procedure.

In one aspect, there is described a skull-spine connection device, comprising a connector connecting a skull connection device to a spine connection device, wherein the connector permits relative movement between the skull connection device and the spine connection device in one or more planes, and wherein the connector is configured to lock the skull connection device and spine connection device relative to one another.

In another aspect, there is described a skull attachment device, comprising a skull attachment member configured to be attached to a skull; a rod fixation assembly configured to attach a rod to the skull attachment member, comprising an inner saddle member having a ledge configured to engage a lower region of the skull attachment member and an extension that extends upwardly through an aperture in the skull attachment member, the extension having a slot sized to receive the rod; an outer saddle member that concentrically fits over the inner saddle member on an upper region of the skull attachment member, the outer saddle member having a slot that aligns with the slot of the inner saddle member and that receives the rod; and a locking member that engages the inner saddle member above the rod when the rod is positioned in the slots, wherein the locking member provides a relative force between the inner saddle member and rod so as to rigidly secure and lock all device members together.

Bone screws are used to fixate the skull attachment device onto the bone. The bone screw can be mounted in the attachment's bore hole in a multi-axial configuration such that the axis of the shank can enter the skull at a variety of angles. Preferably, the screws are placed non-perpendicular to the underlying skull and non-parallel to one another. In this way, a segment of bone is captured between the non-parallel screws and between the individual screw shank and bone surface. These factors will collectively maximize screw resistance to pull-out.

Additional embodiments of the skull attachment device are described, wherein some embodiments will employ plate members to inter-connect the skull and spinal attachments while other embodiments will utilize differing mechanisms (such as Morse taper design) to lock the inter-connector to the attachment devices.

In another aspect, there is described a fastening button for fastening a device to a bone, comprising a base having an engagement surface for engaging a surface of the bone; and a post extending outwardly from the base, an outer surface of the post configured to engage an edge of a hole in the bone, wherein the post is positioned in an off-center location relative to the base.

In another aspect, there is described devices that retain the bone graft, cross-link the different connector components, and/or both. The graft retainers fixate the bone graft in the desired location and adventurously apply a compressive force onto it so as to promote graft incorporation and fusion. The cross-link feature adds stiffness and stability to the whole construct and minimizes the likelihood of component loosening, migration and/or failure.

In another aspect, there is described a skull attachment assembly for spinal fixation, comprising: a skull attachment member adapted to be attached to a skull, the skull attachment member having a central member and first, second, and third arms extending outwardly from the central member, wherein the central body or the arms include at least one borehole adapted to receive a bone fastener for fastening the skull attachment member to a skull.

In another aspect, there is described a skull attachment assembly, comprising a skull attachment member configured to be attached to a skull, the skull attachment member including a rod-based skeleton that supports at least one extension that provides a platform for attaching the skull attachment to a component.

In another aspect, there is described an assembly for retaining a bone graft, comprising a device that rigidly affixes a bone graft in a fusion orientation from a skull to a cervical spine.

In another aspect, there is described a skull-spine connection assembly, comprising a connector adapted to connect at least two elongate spinal interconnectors to one another, the connector being adapted to transition between a locked state and an unlocked state, wherein the connector permits relative movement between the interconnectors throughout one or more planes when in the unlocked state, and wherein the connector immobilizes the interconnectors relative to one another when in the locked state.

In another aspect, there is described a skull attachment assembly, comprising: a skull attachment device having a first borehole for receiving the shank of a bone screw; a protrusion extending upward from the skull attachment device; and at least one fixation assembly mounted on the protrusion, the fixation assembly configured to attach an elongate interconnector to the skull attachment device.

The details of one or more embodiments are set forth in the accompanying drawings and the description below. Other features, objects, and advantages will be apparent from the description and drawings, and from the claims.

### DESCRIPTION OF DRAWINGS

FIG. 1 shows a perspective view of at embodiment of an occipito-cervical fixation system

FIG. 2 shows a perspective, assembled view of a skull attachment device.

FIG. 3 shows the skull attachment device of FIG. 2 in a partially-exploded state.

FIGS. 4A and 4B show cross-sectional views of the skull attachment illustrating the trajectory of bone screws.

FIG. 4C shows a schematic view of a bone structure attached to two occipital attachments.

FIG. 4D schematically shows an occipital attachment attached to the sub-occipital bone.

FIG. 5 shows a cross-sectional view of the skull attachment showing an arrangement of rod fixation assemblies.

FIG. 6 shows another embodiment of the skull attachment.

FIG. 7 shows another embodiment of a skull attachment.

FIG. 8 shows another embodiment of a skull attachment.

FIG. 9 shows another embodiment of a skull attachment.

FIG. 10 shows another embodiment of a skull attachment.

FIGS. 11 and 12 show cross-sectional views of another embodiment of a rod fixation assembly in assembled and exploded states.

FIG. 13 shows yet another embodiment of the rod fixation assembly.

FIG. 14 shows a plan view of another embodiment of a skull attachment.

FIG. 15 shows an exploded view of the skull attachment of FIG. 14.

FIGS. 16 and 17 show cross-sectional views of the skull attachment of FIG. 14.

FIG. 18 shows another embodiment of a skull attachment.

FIGS. 19 and 20 show cross-sectional views of the skull attachment of FIG. 18.

FIG. 21 shows a skull attachment with an alternative embodiment of the rod fixation assembly.

FIGS. 22A and 22B show perspective and top plan views of the skull attachment of FIG. 21.

FIGS. 23A and 23B show cross-sectional views of the skull attachment of FIG. 21.

FIGS. 23C-F show a skull attachment with an alternative embodiment of the rod fixation assembly.

FIG. 24A shows a perspective view of a first embodiment of an occipito-cervical connection system.

FIG. 24B shows an exploded view of the connection system of FIG. 24A.

FIG. 25 shows a cross-sectional view of the connection system.

FIG. 26 shows another embodiment of the connection system.

FIG. 27 shows another embodiment of the connection system.

FIG. 28 shows another embodiment of the connection system.

FIG. 29 shows another embodiment of the connection system.

FIG. 30 shows another embodiment of the connection system.

FIG. 31 shows another embodiment of the connection system.

FIG. 32 shows the connection system of FIG. 31 in an exploded state.

FIG. 33 shows a cross-sectional view of the connection system of FIG. 31.

FIG. 34 shows another embodiment of the connection system.

FIG. 35 shows the connection system of FIG. 34 in an exploded state.

FIG. 36 shows another embodiment of the connection system.

FIG. 37 shows the connection system of FIG. 36 in an exploded state.

FIG. 38 shows another embodiment of the connection system.

FIG. 39 shows the connection system of FIG. 38 in an exploded state.

FIG. 40 shows various views of another embodiment of the connection system.

FIG. 41 shows various views of the connection system of FIG. 40 in an exploded state.

FIG. 42 shows cross-sectional views of the connection system of FIG. 40.

FIG. 43 shows another embodiment of the connection system.

FIG. 44 shows cross-sectional views of the connection system of FIG. 43.

FIG. 45 shows another embodiment of the connection system.

FIG. 46-47 show another embodiment of the connection system.

FIGS. 48A and 48B show cross-sectional views of the connection system of FIG. 46.

FIG. 49 shows another embodiment of the connection system.

FIG. 50 shows the connection system of FIG. 49 in an exploded state.

FIG. 51A shows an exploded view of one embodiment of a combination bone graft retainer and cross-link assembly.

FIG. 51 B shows a cross-sectional view of the attached and locked graft retainer and cross-link assembly.

FIG. 52 shows another embodiment of a graft retainer assembly.

FIGS. 53 and 54 shows a perspective views of the graft retainer of FIG. 52.

FIG. 55 shows an embodiment of a button attachment that includes a post that mates with a locking nut.

FIG. 56 shows views of the locking nut mounted onto the button attachment.

Like reference symbols in the various drawings indicate like or similar elements.

### DETAILED DESCRIPTION

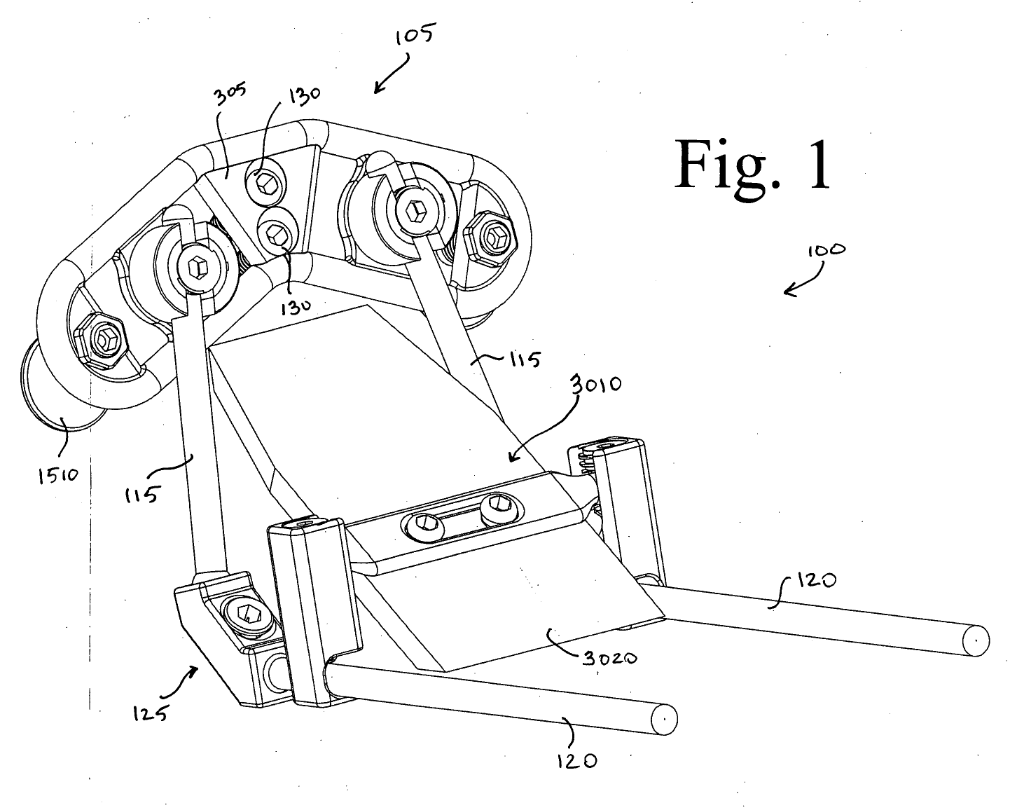

FIG. 1 shows a perspective view of a first embodiment of an occipito-cervical fixation system. The system 100 generally includes at least one skull attachment device 105, at least one interconnecting rod or plate member (depicted as rods 115) that couples onto the skull attachment 105, at least one cervical interconnecting rod or plate member (depicted as rods 120) that couples onto the cervical attachments, and at least one connector 125 that permits relative adjustment of the spatial relationship between the skull and cervical interconnections in one or more planes. The system 100 further includes one or more bone fixators, such as screws 130 or button attachments 1510, that fixate skull attachment 105 onto the underlying bone. Bone screws or other fasteners are similarly used to attach the system onto the cervical bones (not shown). A graft retainer and cross-link assembly 3010 fixates a graft 3020 onto the underlying bone, as will be described in detail below. The graft retainer is adapted to rigidly affix a graft in a desired orientation relative to the skull and the cervical spine.

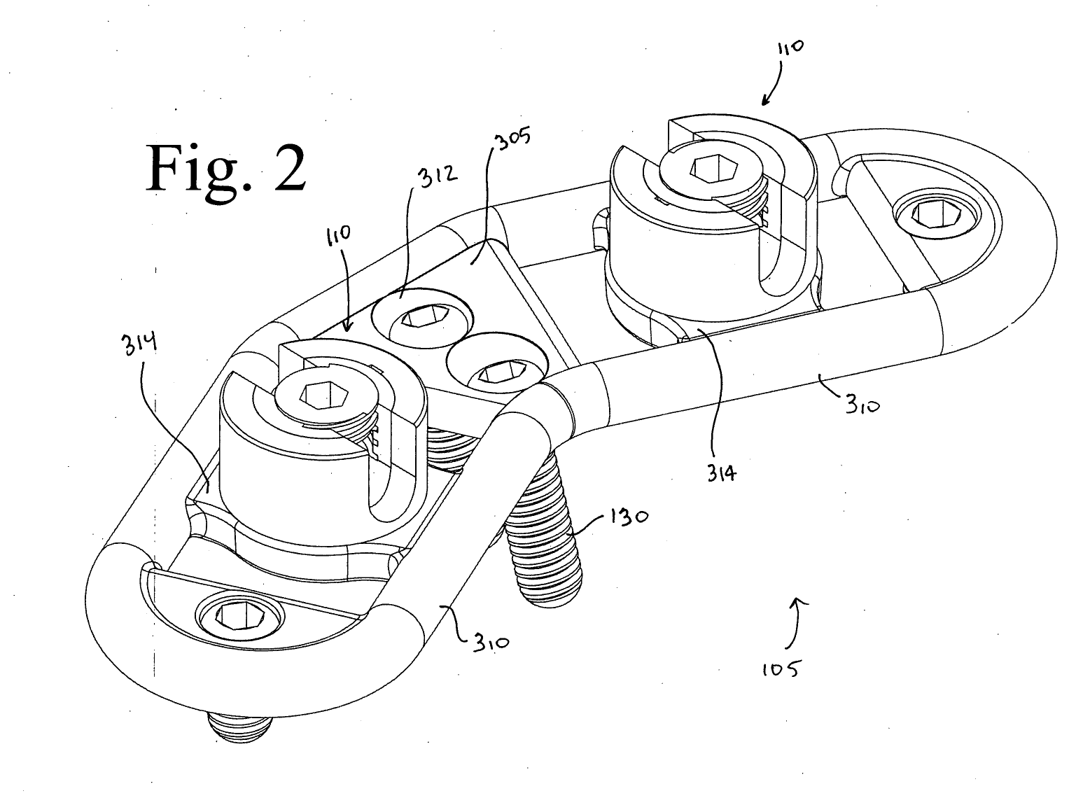

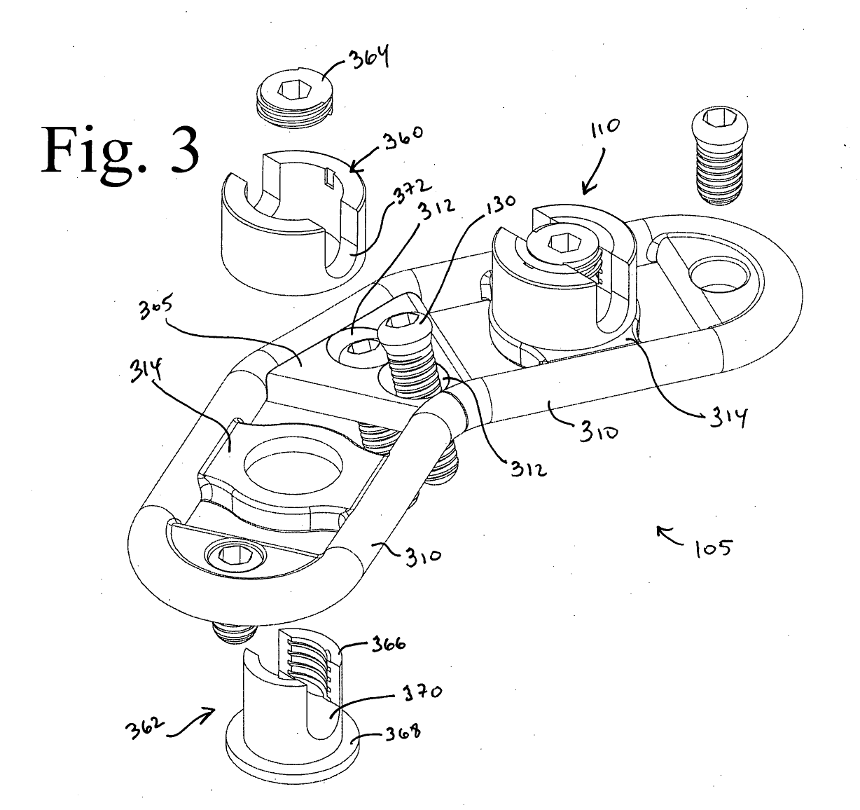

FIG. 2 shows a perspective, assembled view of a skull attachment device 105. FIG. 3 shows the skull attachment device in a partially-exploded state. The skull attachment 105 is configured to be attached to the skull, such as onto the sub-occipital bone of the skull. The skull attachment 105 is made of a rod-based super-structure that forms a skeletal core of the device. That is, one or more rods or elongate members are formed into a shape that forms a structural framework, skeletal structure or skeleton 310 of the attachment 105. One or more extensions arise from skeleton 310 and these extensions may point into the central cavity of skeleton 310 or away from it. The extensions can be platforms that provide a location where the skull attachment can be attached or coupled to other devices, such as bone screws or rod fixation assemblies. Further, the extensions may be rod-like, plate-like or a combination thereof.

In attachment 105, an extension 305 is situated in the central aspect of the device and spans a central cavity of skeleton 310, connecting an upper segment of skeleton 310 with a lower segment. Extensions that connect different portions of skeleton 310 will necessarily increase the rigidity and torsional stability of the device. In addition, they will provide a platform through which additional members may be coupled to attachment 105. Central extension 305 contains one or more boreholes 312 that receive bone screws 130 or other fasteners. Each borehole 312 is configured so that a bone screw received therein can be preferably placed in a multi-axial orientation relative to extension 305. That is, the shank of bone screw 130 may be placed at any of a variety of angles relative to extension 305 and the underlying bone. This is accomplished by using any of a variety of well known complimentary screw head and borehole configurations (such as a conical head in a cylindrical hole, spherical head in a spherical hole, and the like) that can provide such a feature.

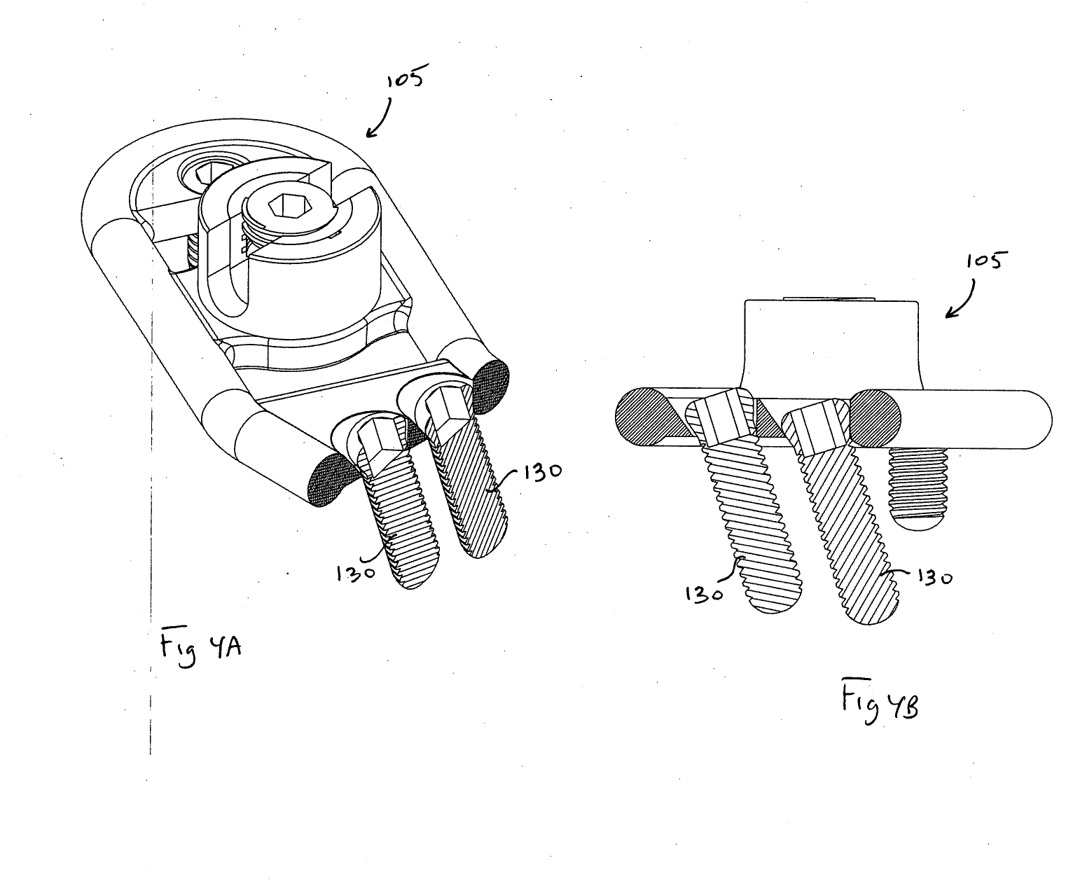

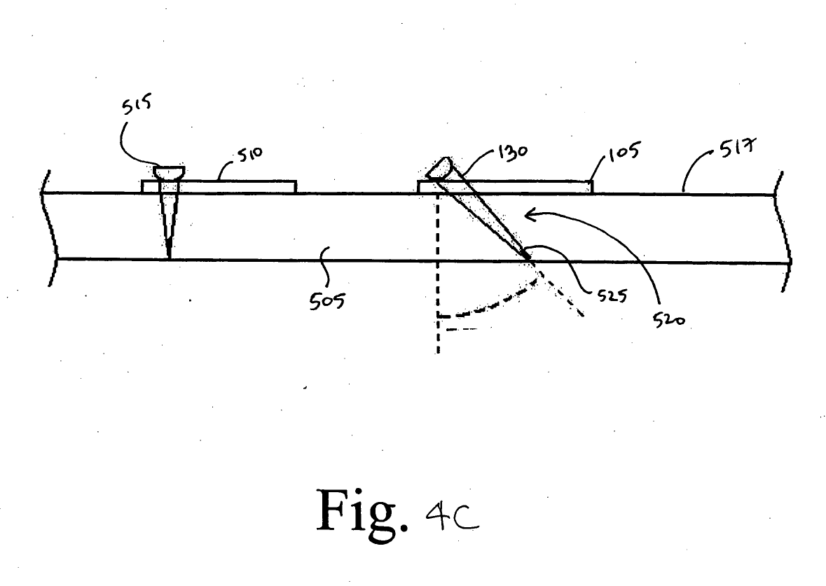

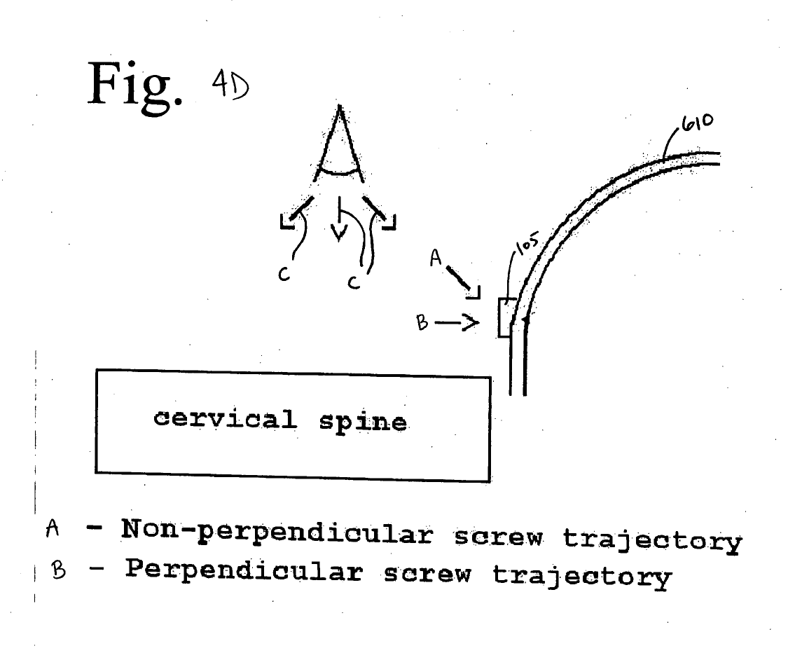

In one embodiment, the axis of each borehole 312 is angled to facilitate bone screw placement into the underlying bone at a non-perpendicular angle. FIGS. 4A and 4B show cross-sectional views of the skull attachments 105 illustrating the trajectory of the bone screws 130. During device implantation, the bone screws are preferably placed non-perpendicular to the bone surface and in non-parallel trajectories to one another. In an alternative embodiment, the borehole trajectory is perpendicular to the top surface of extension 305 and the surgeon guides the poly-axial screws into the preferred trajectory of non-perpendicular placement relative to the bone surface and in non-parallel trajectory relative to one another.

An advantage of screw placement in a non-perpendicular trajectory is shown in FIG. 4C. The diagram illustrates a schematic view of bone 505 with skull attachment 105 having a non-perpendicular bone screw trajectory relative to the bone surface and a skull attachment 510 having a perpendicular screw trajectory. The non-perpendicular trajectory of bone screw 130 permits use of a longer screw than the perpendicular trajectory of screw 515. Moreover, a wedge-shaped region 520 of bone is interposed between the shank 525 of screw 130 and the bone surface 517. The wedge shaped region 520 would have to be dislodged before the angled screw 130 can be avulsed from the occipital bone. These two factors work synergistically to significantly increase screw resistance to pull-out in a manner independent of screw design.

An additional advantage of the poly-axial bone screw configuration is described with reference to FIG. 4D, which schematically shows a skull attachment 105 attached to the sub-occipital bone 610. The axial trajectory of a poly-axial bone screw is represented by the arrow labeled “A”. This bone screw enters the bone at a downward angle (relative to FIG. 4D) that is non-perpendicular to the bone. The axial trajectory of a bone screw that enters the bone at a right angle is represented by the arrow labeled “B” in FIG. 4D. In addition, the surgeon's typical lines of vision during surgery are also shown and labeled C in FIG. 4D. The bone screw with the trajectory A is more inline with the surgeon's line of vision than the bone screw with the trajectory B. This makes the procedure technically easier and decreases the likelihood of incorrect screw placement. It should be appreciated that where the occipital attachment includes more than one bone screw, the screws are preferably placed non-parallel to one another. This provides a “crossed-screw” configuration and forms an additional wedge of bone between the screw shafts, thereby further strengthening the fixation of the occipital attachment to the bone.

Bone screws 130 can be locked relative to the central body 305, such as by using threads on the screws that mate with corresponding threads within the central body 305. In another embodiment, a locking mechanism can be positioned on the central body, wherein the locking mechanism engages the heads of the bone screws to lock the bone screws relative to the central body 305. In addition to these, there are numerous known mechanisms through which the screws can be retained relative to body 305. Some of these mechanism and methods are illustrated in U.S. Pat. Nos. D440311S, D449692S, 5,364,399, 5,549,612, 5,578,034, 5,676,666, 5,681,311, 5,735,853, 5,954,722, 6,039,740, 6,152,927, 6,224,602, 6,235,034, 6,331,179, 6,454,769, 6,599,290, 6,602,255, 6,602,256, 6,626,907, 6,652,525, 6,663,632, and 6,695,846. It is understood that any of these and/or other mechanisms for screw retention may be applied to the devices disclosed in this application and that these variations would fall within the scope of the present invention. Fixation of the bone screw to the skull attachment 105 will further increase the screw resistance to pull-out.

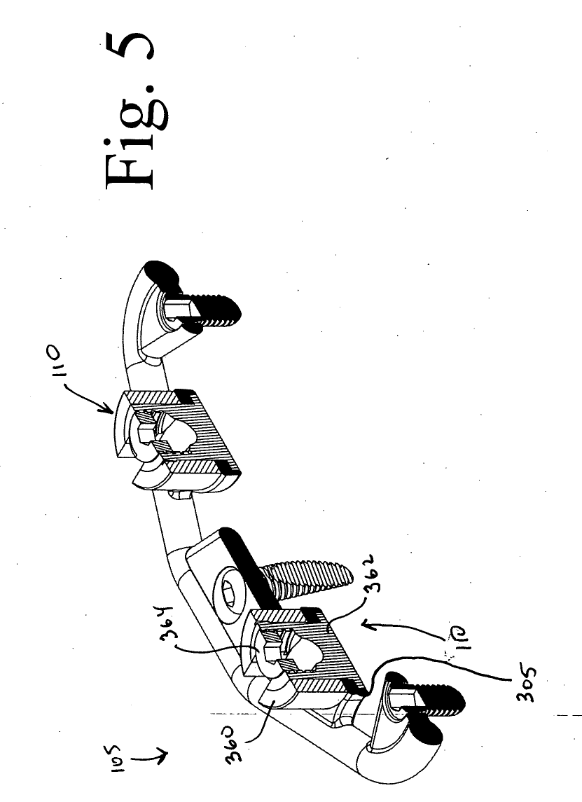

With reference to FIGS. 2 and 3, at least one extension 314 is contained in the skull attachment 105. A rod fixation assembly 110 is affixed onto extension 314, such that assembly 110 removably secures an elongate interconnector, such as a rod 115 (FIG. 1), to the skull attachment 105. FIG. 2 shows the rod fixation assemblies 110 in the assembled state, while FIG. 3 shows one of the rod fixation assemblies 110 in an exploded state. FIG. 5 shows a cross-sectional view of the skull attachment 105 showing the arrangement of the rod fixation assemblies 110.

As best shown in FIGS. 3 and 5, each rod fixation assembly 110 includes an outer saddle member 360, an inner saddle member 362, and a locking nut 364. The inner saddle member 362 includes a cylindrical protrusion 366 that extends upwardly from a base having an annular ledge 368. The protrusion 366 is sized to fit through an aperture in the extension 314, although the ledge 368 has a transverse dimension that is larger than the dimension of the aperture. The transverse dimension of the ledge 368 is sized to sit within a seat or indentation (shown in FIG. 5) on the underside of the extension 314. A slot 370 is formed within the protrusion 366 for receiving the rod 115. Threads are disposed in an inner aspect of the protrusion 366 for mating with corresponding threads of the nut 364. The outer saddle member 360 includes a cylindrical wall having a slot 372 extending therethrough. The slot 372 is configured to align with the slot 370 on the inner saddle member 362 when the inner saddle member 362 is positioned within the outer saddle member 360. The interactions of protrusion 369 of inner saddle member 362 and indentation 361 of outer saddle member 360 keep slots 370 and 371 of the two saddle members aligned and assembly 110 in the assembled state.

When the rod fixation assembly 110 is in the assembled state, the inner saddle member 360 is concentrically positioned within the outer saddle member 362 with the extension 314 sandwiched therebetween. The ledge 368 of the inner saddle member 362 engages a seat on the underside of the extension 314. The protrusion 366 extends upwardly through the aperture in extension 314. The locking nut 364 is positioned within the inner saddle member 362 such that outer threads on the nut engage inner threads in the inner saddle member 362. The locking nut 364 secures the rod 115 in place within the slots 370 and 372. The bottom of the slot 370 within the inner saddle member 362 is lower than the corresponding slot 372 within the outer saddle member 360 such that the lower surface of the rod 115 contacts the outer saddle member. The upper surface of the occipital rod 115 contacts the lower surface of the locking nut 364 to secure the rod to the rod fixation assembly 110.

Attachment 105 may also contain one or more extensions 306 that have at least one borehole. These extensions would serve as additional platforms to which screws or other fasteners can be coupled and used to anchor attachment 105 to the underlying bone. In FIG. 1, a bone fixation button is illustrated attached to the borehole of extension 306 while FIG. 3 shows the use of bone screws. As in extension 305, the screw trajectory is preferably multi-axial and can be placed into the underlying bone at a variety of angles. In application, the screws of extension 305 are preferably placed in a non-perpendicular trajectory relative to the bone surface while the screws of extension 306 are preferably placed in a substantially more perpendicular trajectory. The placement of different bone screws in non-parallel orientation produces a “crossed screw” configuration which maximizes the resistance to screw pull-out.

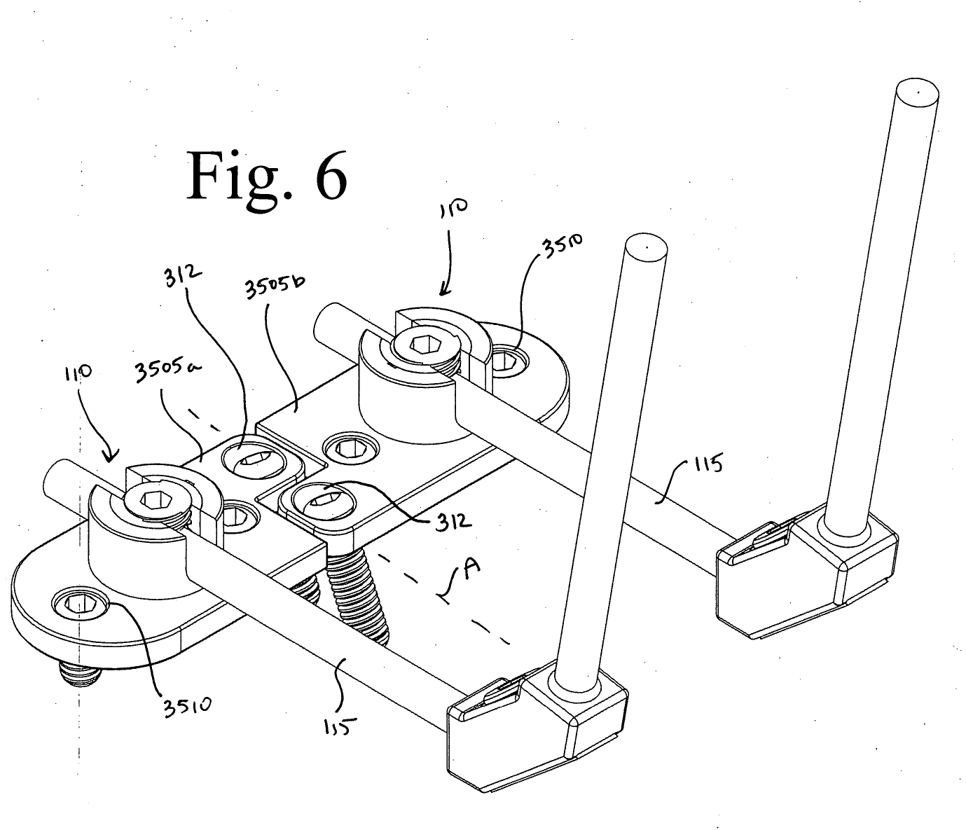

FIG. 6 shows another embodiment of the skull attachment, wherein two or more plate members 3505a and 3505b are sized and shaped such that that they abut or engage one another in a complementary fashion along a central axis A. For example, the plate members 3505 can each have complementary shaped and positioned protrusions that align or interlock with one another. Each plate member 3505 includes a central segment with borehole 312 that receive a bone screw, wherein boreholes 312 of two complementary plates may be aligned as shown in FIG. 6. The bone screws are mounted in a multi-axial configuration such that the axis of the shank can enter the skull at variety of angles. The borehole trajectory is preferably angled relative to the top surface of the central segment so as to guide the screws trajectory is non-perpendicular to the underlying skull. Each plate member 3505 includes an extension segment that protrudes away form the central segment at any desired angle. The extension has one or more lateral boreholes 3510 that are adapted to receive a bone screw or other bone fasteners. The placement of different bone screws in non-parallel orientation produces a “crossed screw” configuration which increases resistance to screw pull-out. In addition, each plate member 3505 includes borehole that houses a rod fixation assembly 110. Assembly 110 is configured to attach the respective plate member 3505 to a respective rod 115.

As previously described, rod fixation assembly 110 contains an inner saddle member having a ledge configured to engage the lower aspect of borehole 3506 of plate member 3505 and an extension that extends upwardly through borehole 3506, the extension having a slot sized to receive the rod; an outer saddle member that concentrically fits over the inner saddle member and rests upon the upper surface of plate member 3505, the outer saddle member having a slot that aligns with the slot of the inner saddle member and that receives the rod; and a locking member that engages the inner saddle member above the rod when the rod is positioned in the slots, wherein the locking member provides a relative force between the inner saddle member and rod so as to rigidly secure and lock all assembly members together and to the plate.

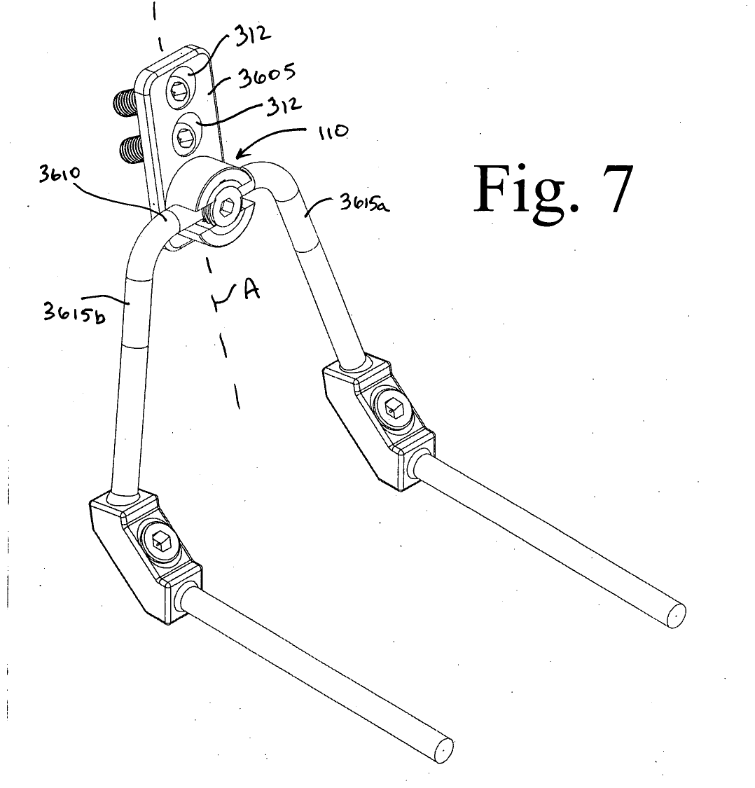

FIG. 7 shows another embodiment of a skull attachment that includes a single plate member 3605 that extends along a central axis A, which can be aligned with the midline of the sub-occipital bone. In the illustrated embodiment, the plate member 3605 is rectangular, although the plate member 3605 can have various shapes. The plate member 3605 includes one or more boreholes 312 for receipt of a bone screw or other bone fixation devices. The plate member 3605 also includes a rod fixation assembly 110. While the borehole(s) 312 and rod fixation assembly 110 are depicted along the central axis A, It should be appreciated that the relative positions of the boreholes 312 and the rod fixation assembly 110 along the axis A can be varied and need not be co-linear. The skull attachment of FIG. 7 includes one or more rod fixation assembly 110. It attaches to a rod 3610 that extends laterally outward in both directions relative to the axis A and curves downward at both ends to form a pair of rods 3615a and 3615b.

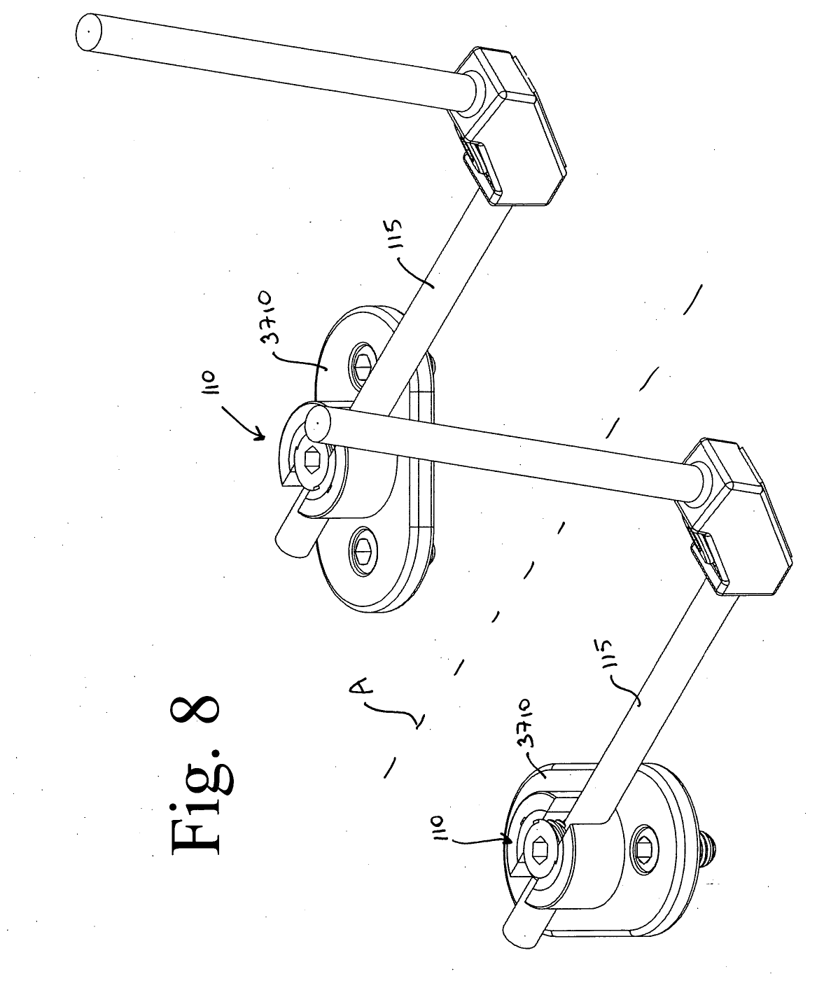

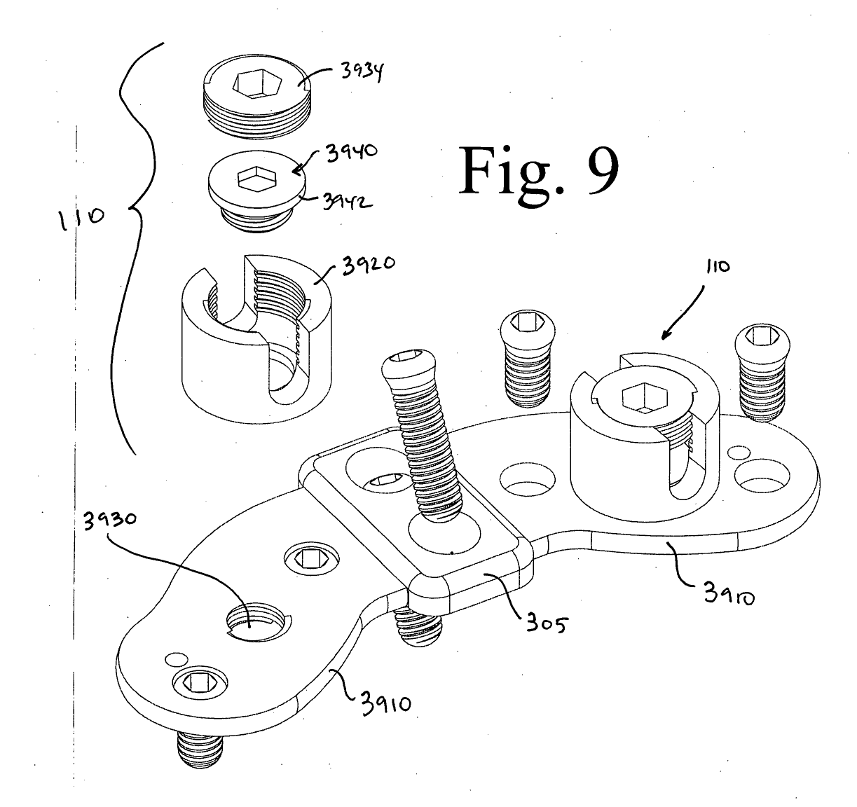

FIG. 8 shows another embodiment of a skull attachment. Plate members 3710 are positioned on opposite lateral sides of the central axis A. Each plate member 3710 includes one or more boreholes for receiving bone screws or other fasteners. In addition, each plate member 3710 includes a respective rod fixation assembly 110 for attachment to a rod 115. In use, each plate member 3710 is attached to a region of the skull, such as on opposite sides of the midline of the sub-occipital bone. This embodiment is especially useful in those patients who have undergone surgical resection of a portion of the skull (such as the midline segment of the sub-occipital skull) and require one or more skull attachments of relatively compact size.

FIG. 9 shows another embodiment of the skull attachment. In this embodiment, two extensions 3910 extend outward from the central body. Each extension 3910 includes a rod fixation assembly that can differ from that previously described in other embodiments. A single saddle member 3920 is attached to borehole 3930 using a screw 3940. A shoulder 3942 limits advancement of screw 3940 into borehole 3930 so that, when the screw is fully seated, the saddle member 3920 can still rotate about screw 3940. Further, the screw is sufficiently wedged and tightened into borehole 3930 so that it can not be loosened with any force it is likely to encounter in actual use. When the rod is placed within saddle member 3920, a locking nut 3934 can be used to immobilize it. Locking nut 3934 is tightened and advanced until the rod is immobilized between the nut 3934 and the screw 3940. Unlike the locking mechanism of prior embodiments, saddle member 3920 remains freely rotatable relative to screw 3940 even after immobilization of the rod. However, since the immobilized rod rests within the rod-receiving slot of member 3920, the saddle member is effectively prevented from rotation when locking nut 3924 is tightened.

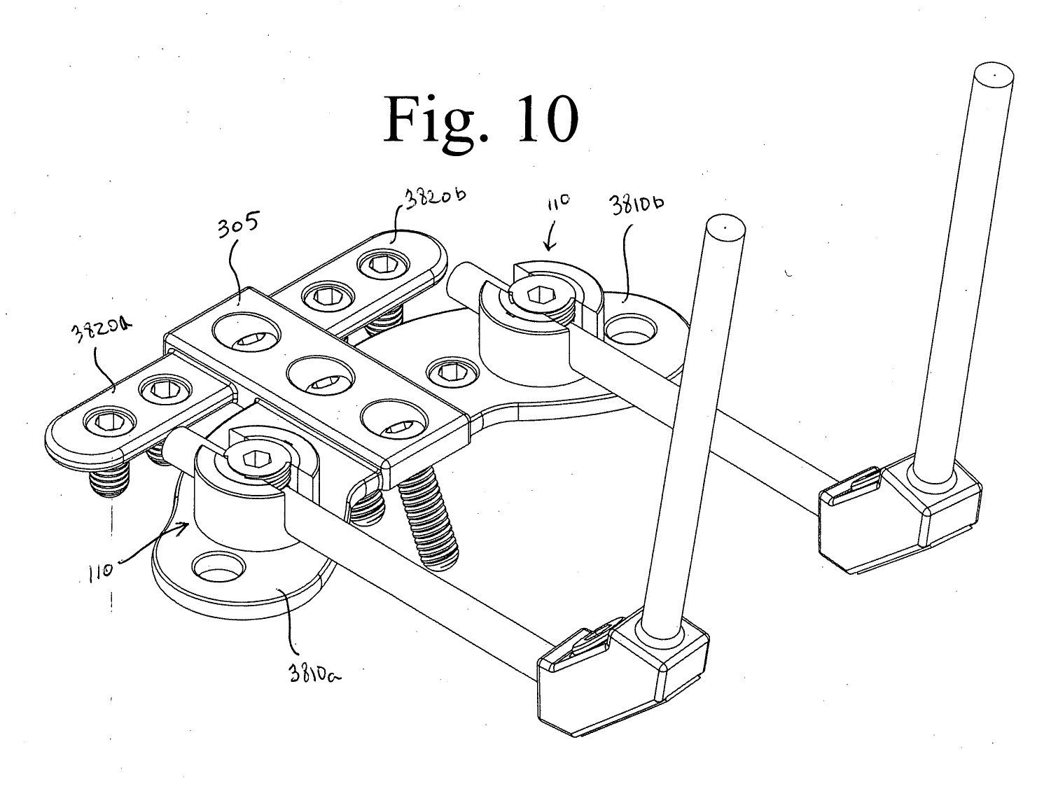

FIG. 10 shows yet another embodiment of the skull attachment. This embodiment includes a central body 305 having one or more boreholes 312 for bone screws. A first set of arms or extensions 3810a and 3810b extend laterally outward from the central body 305. A second set of extensions 3820a and 3820b also extend laterally outward from the central body 305. The extensions 3810 and 3820 are shown as having a plate-like configuration, although it should be appreciated that the extensions can be formed of curvilinear rods, plates or combinations thereof. Thus, three or more extensions extend outwardly from the central body.

The central body and/or one or more extensions include a respective rod fixation assembly 110 for attaching the skull attachment onto rod(s) 115. For example, the rod fixation assemblies can be placed on either extensions as depicted in FIG. 10. Each of extensions 3810 and 3820 include one or more boreholes for the attachment of bone screw or other bone fasteners. Extensions 3810 and 3820 are positioned relative to the central body 305 so as to provide additional fixation for the attachment when affixed to the sub-occipital bone or other skull segment. While depicted as extending from central body 305 at a particular angle, it should be appreciated that the extensions 3810 and 3820 can extend outward from body 305 at various angles and may be further designed so that the angle can be varied by the surgeon at the time of implantation.

The skull attachment of FIG. 10 thus includes a first region comprised of the central body 305, and a second region comprised of three or more extensions that extend outwardly from the central body 305. While each extension is depicted as emerging from a separate point on the central body 305, it is understood a single attachment point on the central body may give rise to two or more extensions, whereby the single point of attachment on the central body produces a single extension that can divide further into two or more extensions. Each of the central body and extensions contains one or more boreholes that receive bone screws or other bone fasteners. In addition, each of the central body and extensions may contain one rod fixation segment so that the totality of the skull attachment device contains at least one rod fixation segment. Alternatively, if plate inter-connectors are used to connect the skull attachment to the cervical attachment(s), then the totality of the skull attachment device contains one or more point of attachment for the plate inter-connectors.

The bone screw can be mounted in the attachment's bore hole in a multi-axial configuration such that the axis of the shank can enter the skull at a variety of angles. Preferably, the screws are placed non-perpendicular to the underlying skull and non-parallel to one another. A non-perpendicular trajectory is particularly desirable with placement of the bone screws that engage central body 305.

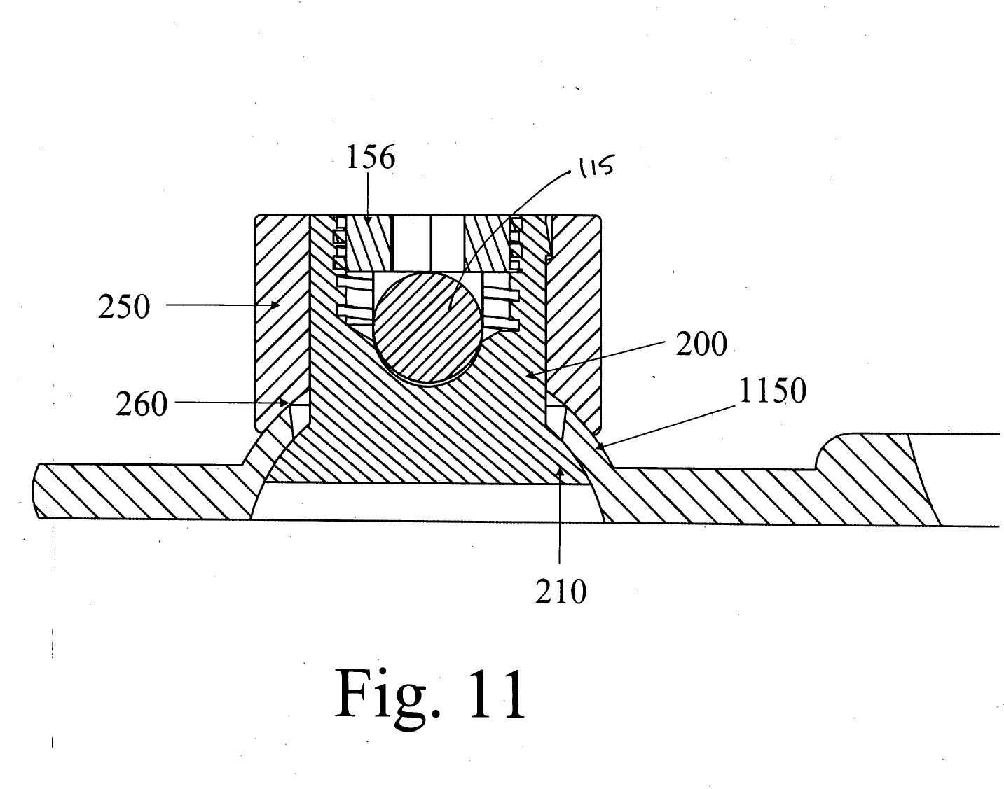

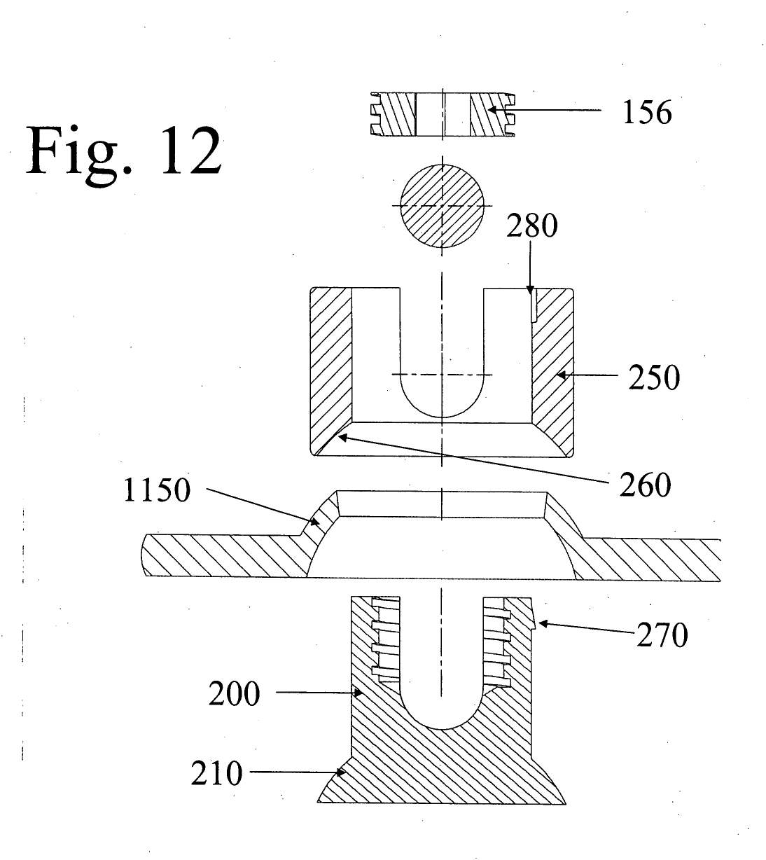

FIGS. 11 and 12 show cross-sectional views of another embodiment of the rod fixation assembly in the assembled and exploded states. This embodiment is configured to rotate about its central axis prior to tightening. In addition, this embodiment can be swiveled and oriented at various angles relative to the top surface of the skull attachment 105.

The rod fixation assembly 110 of FIGS. 11 and 12 includes an outer saddle 250, an inner saddle 200, and a lock nut 156. The assembly removably couples to the rod 115. The skull attachment 105 has a rounded protrusion 1150 with a borehole extending therethrough. The protrusion 1150 has an upper rounded surface and a lower rounded surface. The outer saddle has a lower, rounded surface 260 that is shaped to complement the shape of the upper surface of the rounded protrusion 1150. The inner saddle 200 has a flange 4430 with an upper, rounded surface that complements the lower rounded surface of the protrusion 1150.

In use, the inner saddle 200 is inserted upwardly through the borehole in the protrusion 1150 such that the rounded upper surface inner saddle 200 abuts the rounded lower surface of the protrusion 1150. The outer saddle 250 is lowered onto the protrusion 1150 such that the lower rounded surface 260 of the outer saddle 250 abuts the upper rounded surface of the protrusion 1150. The inner saddle 200 has a locking member 270 (FIG. 12) that locks with an indentation 280 on the outer saddle 250 to lock the assembly together. The complementary round shapes of the protrusion surfaces and the surfaces of the inner and outer saddles permits the saddles to be swiveled relative to the protrusion 1150.

A rod 115 is then lowered into a slot in the outer saddle 250 and inner saddle 200 and the locking nut 156 tightened onto the rod to lock all the components together. The slot of the outer saddle 250 has a bottom surface that is slightly higher than a bottom surface of the slot in the inner saddle 200. Thus, the inferior aspect of the rod 115 is pressed against the bottom surface of the slot in the outer saddle 250. The rod does not engage the bottom surface of the slot in the inner saddle. In this way, the protrusion 1150 of the skull attachment is locked between the downward displacement of the outer saddle and the upward displacement of the inner saddle and the device is locked in place.

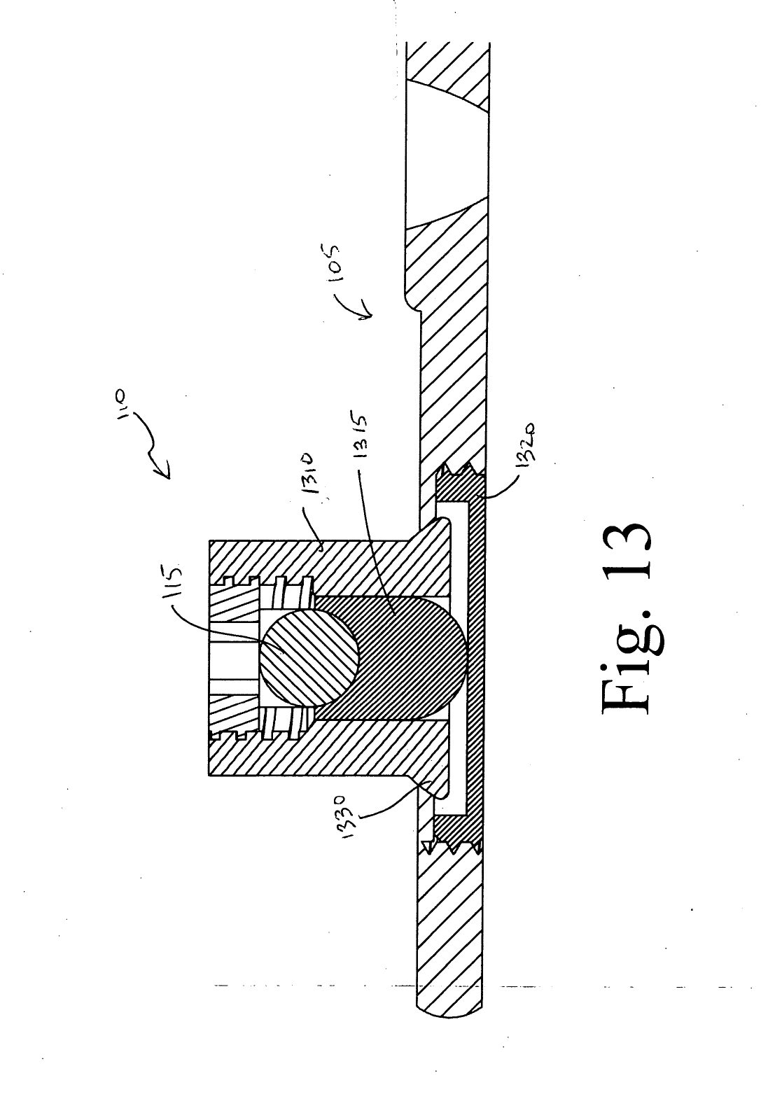

FIG. 13 shows yet another embodiment of the rod fixation assembly 110. This embodiment is also configured to rotate about a central axis and also swivel to form an angle relative to the top surface of the skull attachment 105. The assembly includes an outer member 1310, an inner member 1315, a lower locking member 1320, and a lock nut 4525. The inner member 1315 has a rounded lower surface that abuts an upper surface of the lower locking member 1320. The lower locking member 1320 engages the skull attachment 105 using threads. The rod 115 is secured between the outer member 1310 and the inner member 1315. The outer member 1310 has a flange 1330 with a rounded surface that can swivel relative to the plane of the skull attachment 105. Thus, the top surface of the saddle 1310 can angulate relative to the top plane of the skull attachment 105.

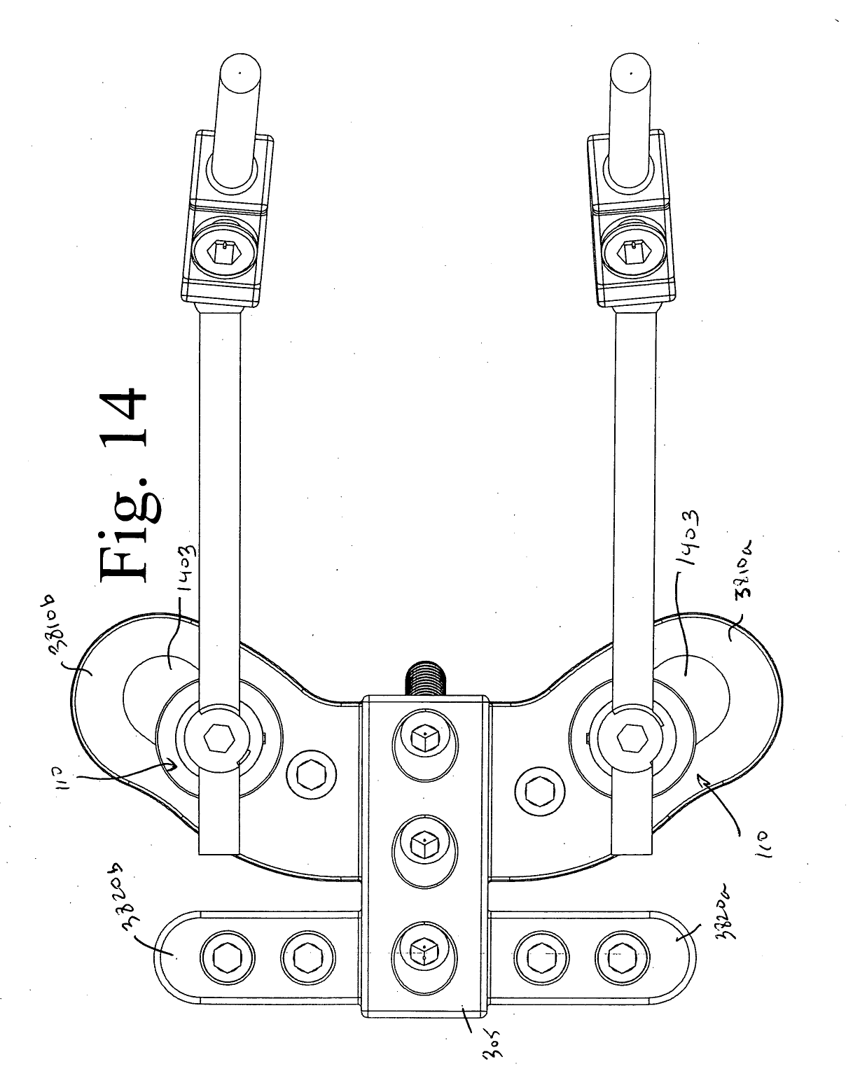

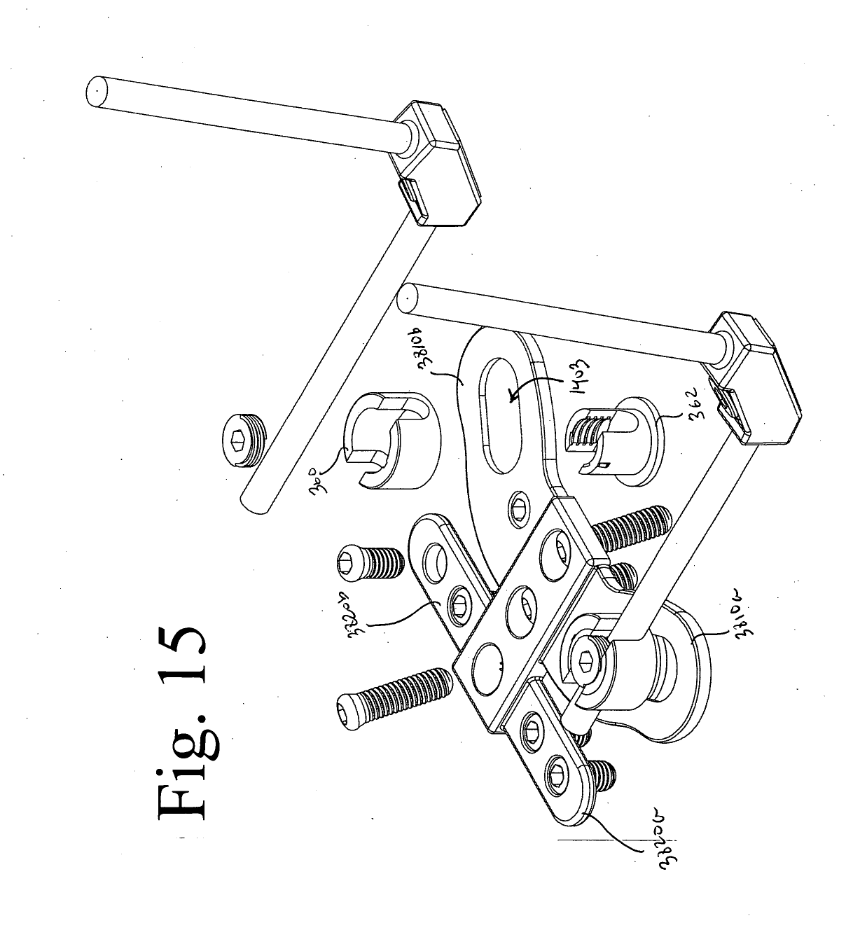

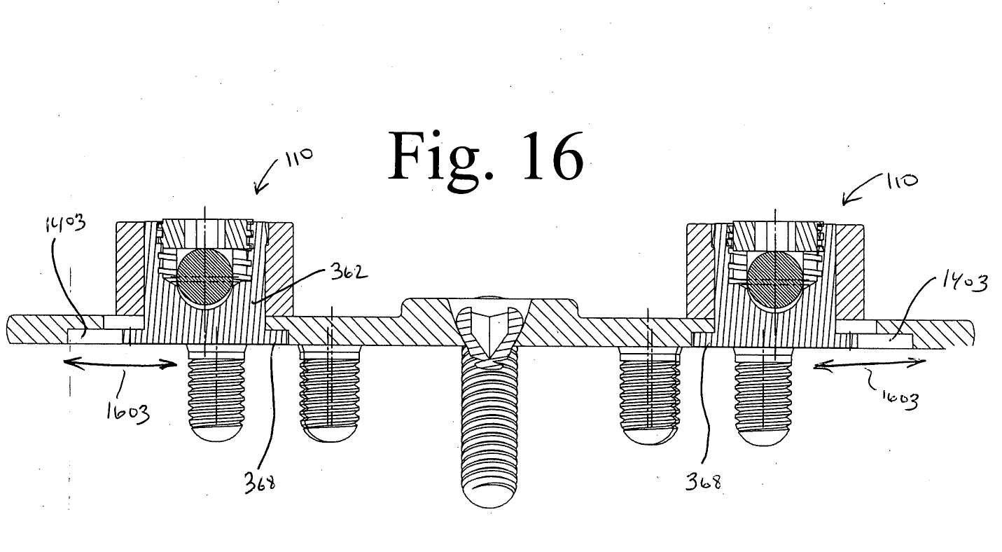

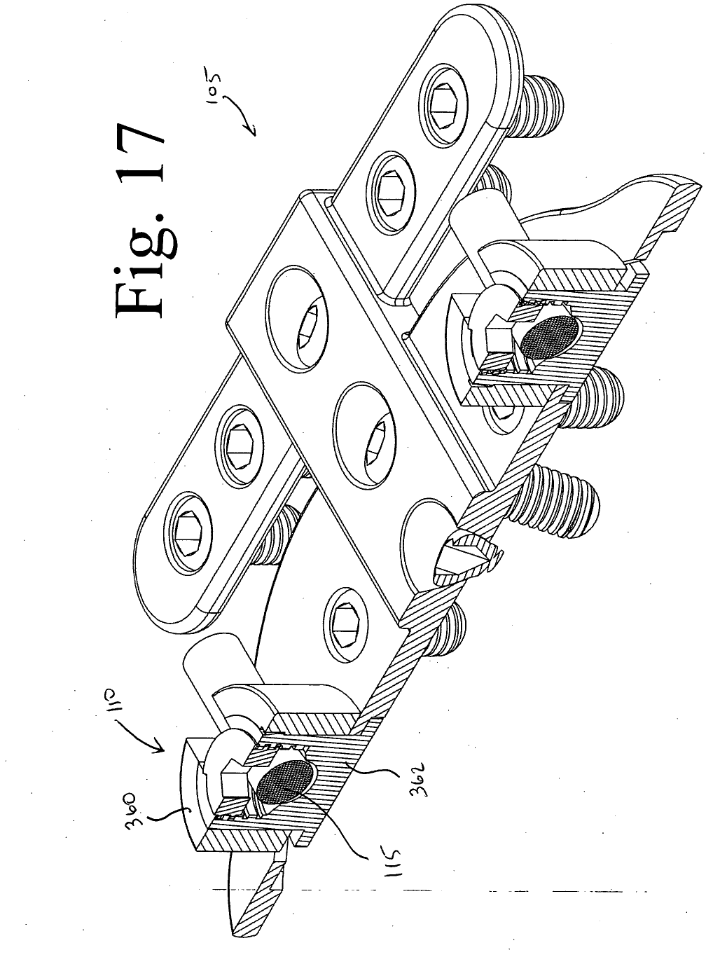

FIG. 14 shows a plan view of another embodiment of a skull attachment that is similar to the skull attachment of FIG. 10. FIG. 15 shows an exploded view of the skull attachment of FIG. 14. FIGS. 16 and 17 show cross-sectional views of the skull attachment of FIG. 14. In this embodiment, the rod fixation elements 110 are positioned in elongate apertures 1403. The rod fixation elements 110 each include an inner saddle member 362 and an outer saddle member 360 of the type described above with reference to FIG. 3. The rod fixation elements 110 can be positioned at various locations along the length of the elongate apertures 1403 and immobilized at a desired location. Thus, as shown in FIG. 16, the rod fixation elements 110 can be movably positioned (as represented by the arrows 1603) along the elongate apertures 1403. An indented seat on the bottom side of the extensions 3810 is sized to accommodate the annular ledge 368 on each of the inner saddle members

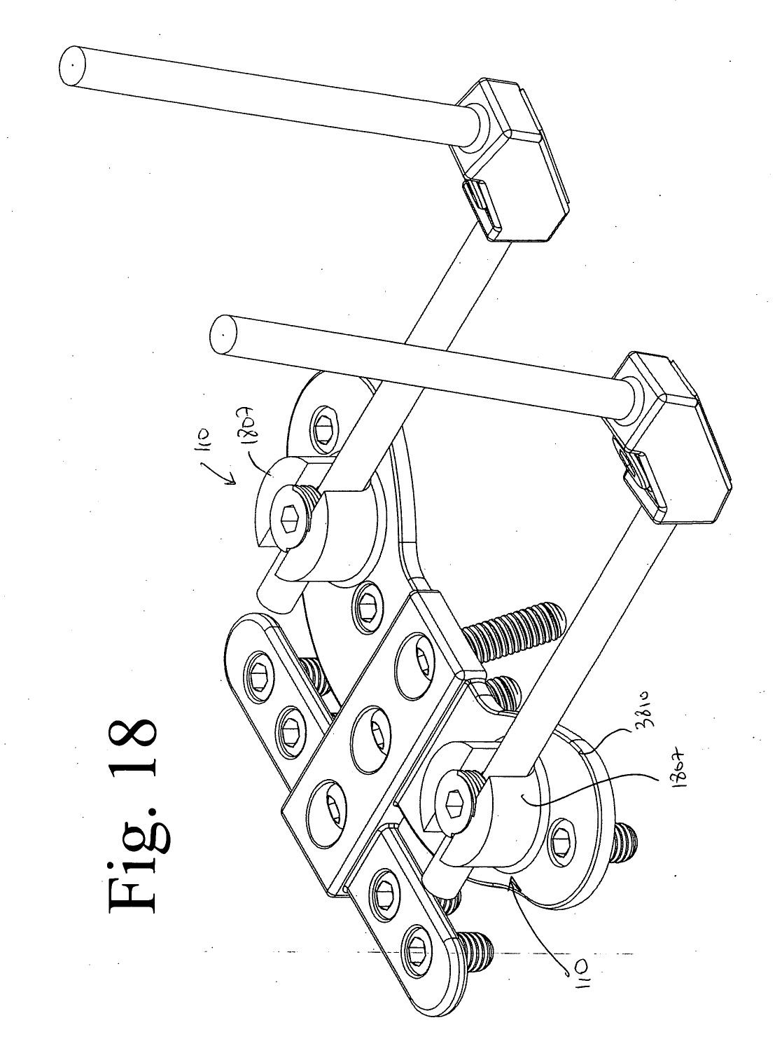

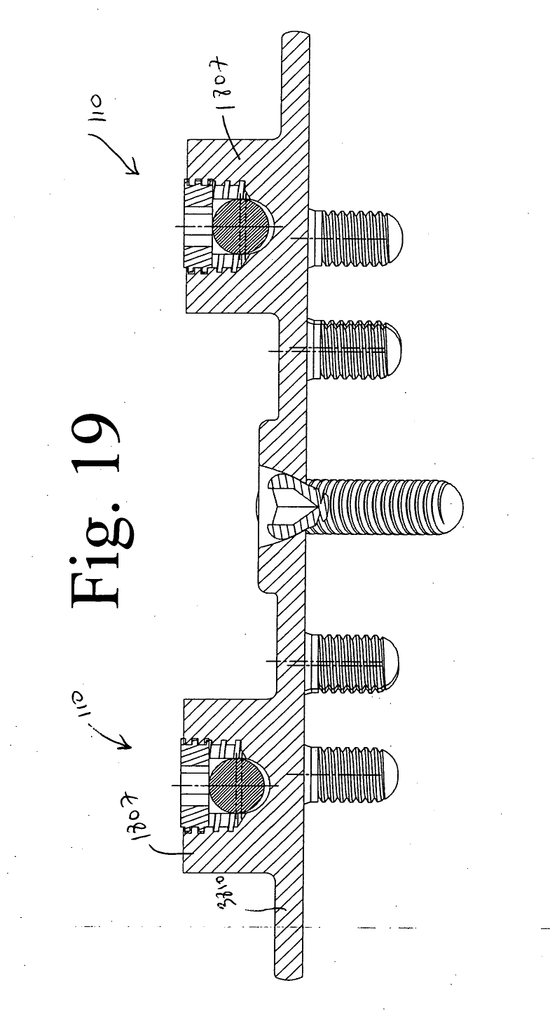

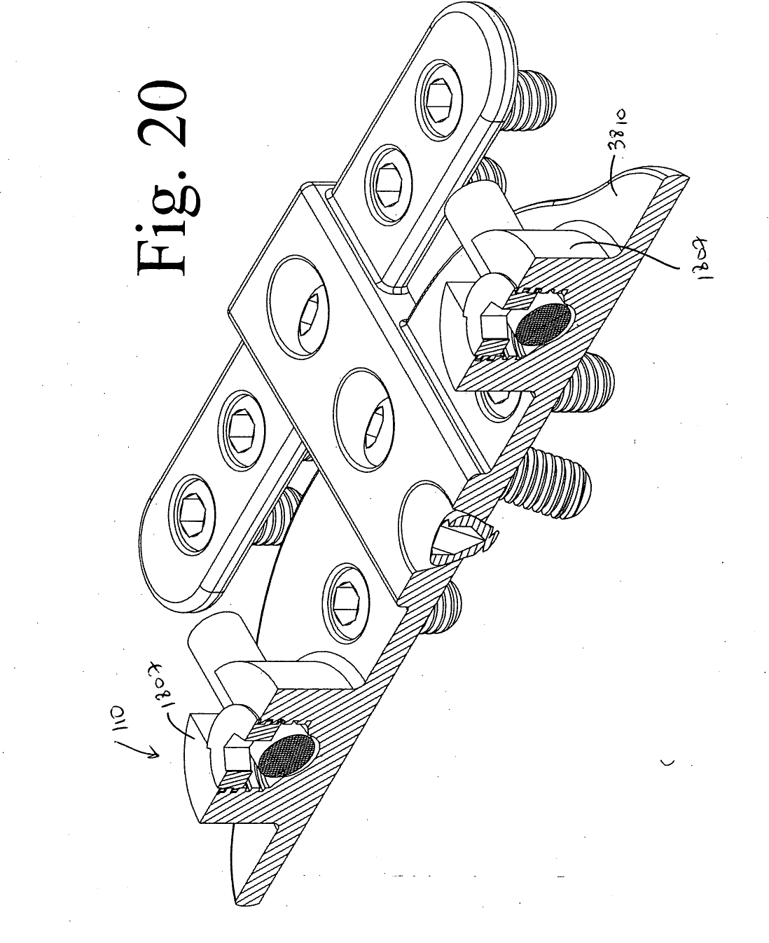

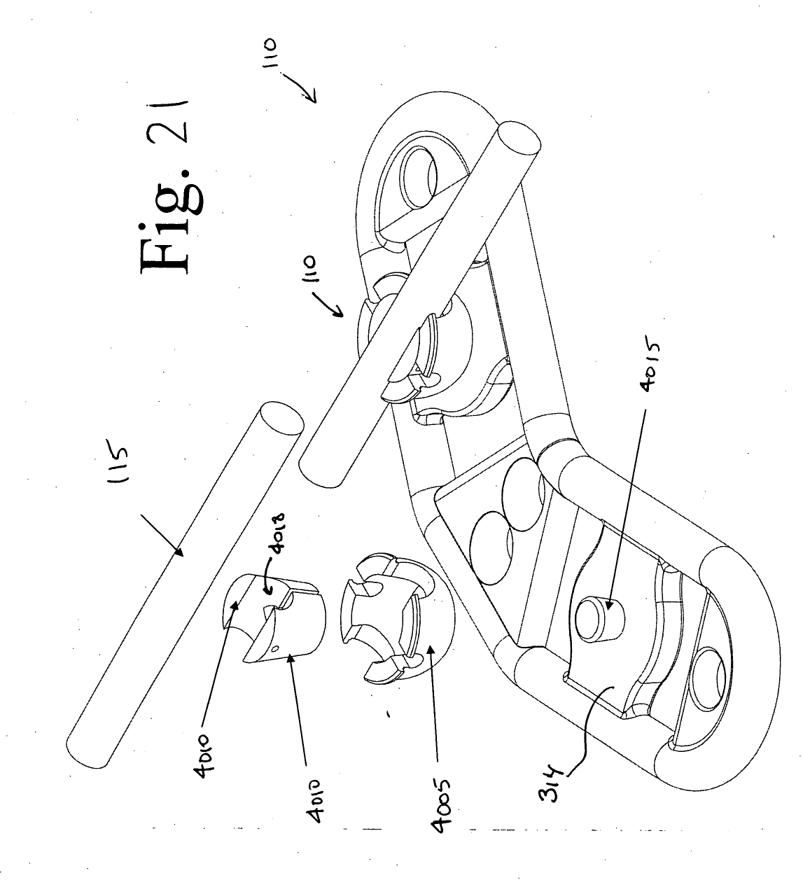

FIG. 18 shows another embodiment of a skull attachment of the type described above with reference to FIG. 14. FIGS. 19 and 20 show cross-sectional views of the skull attachment of FIG. 18. In this embodiment, the rod fixation elements 110 include a saddle member 1807 that is integrally formed with the skull attachment. The term “integrally formed” or “integrally attached” means that the pieces or segments are formed of a unitary or single member, although two segments can be integrally attached or formed and made of two separate materials. This differs from two segments or pieces that are removably attached to one another. The saddle member 1807 extends upwardly from an upper surface of each of the extensions 3810. Each of the saddle members 1807 includes a slot that receives a rod. A locking member can be advanced into the saddle member 1807 to lock the rod in place.

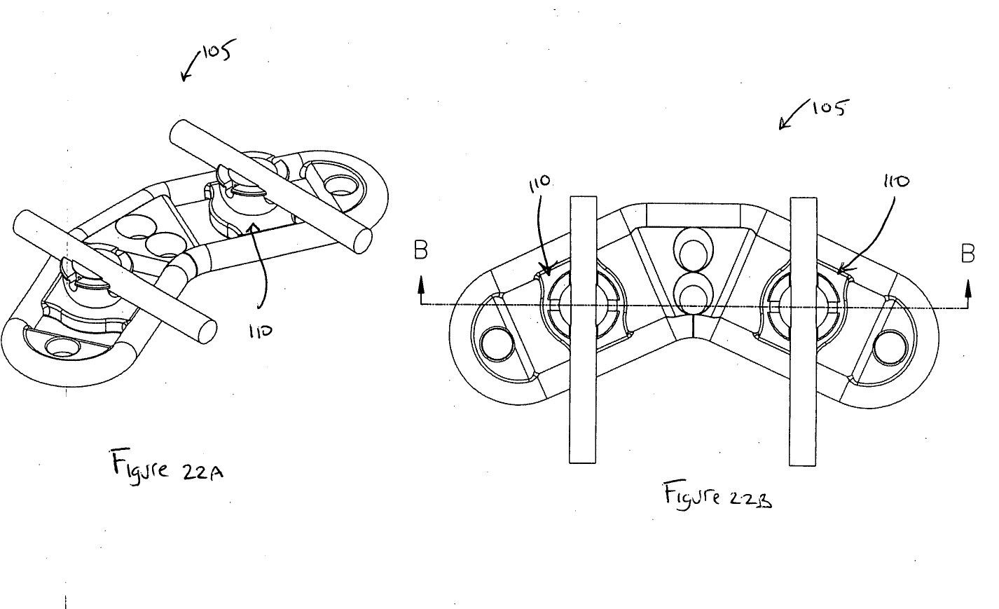

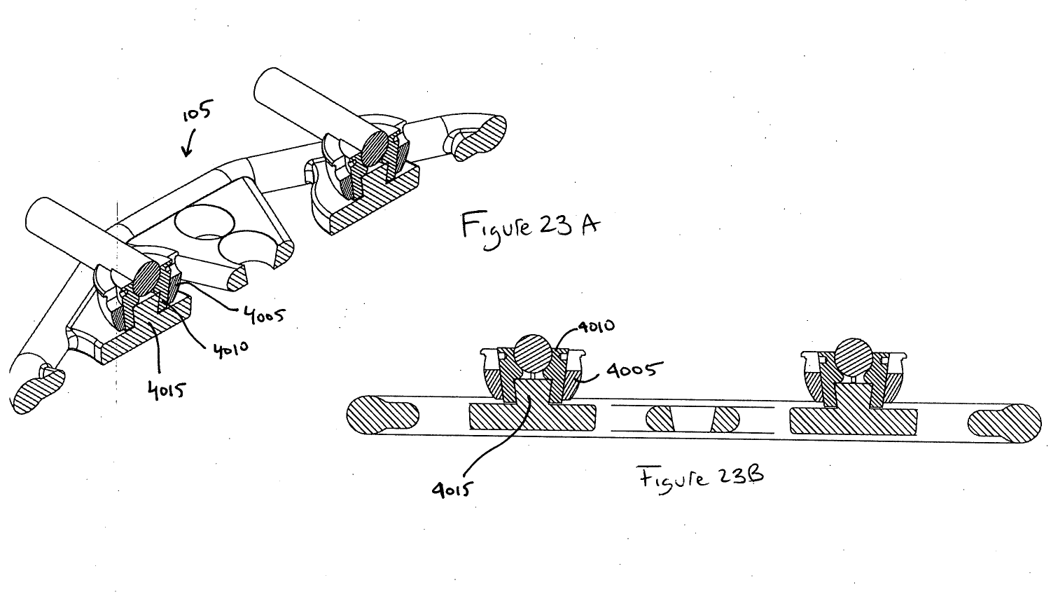

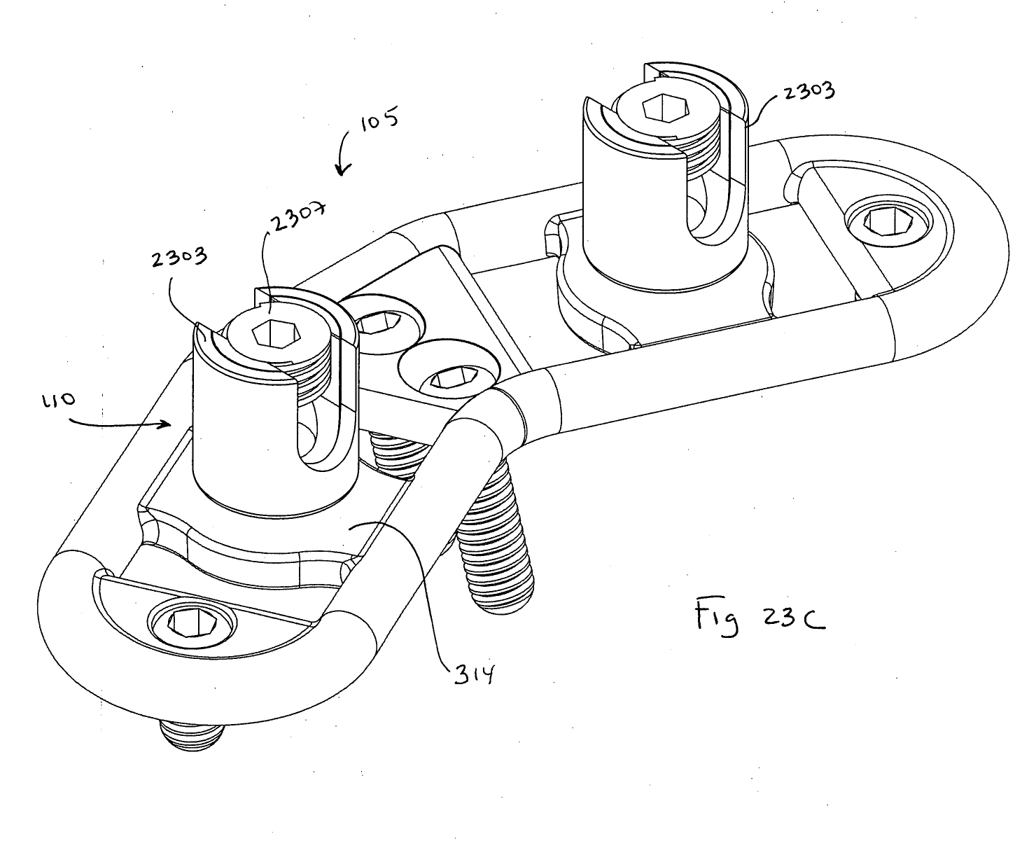

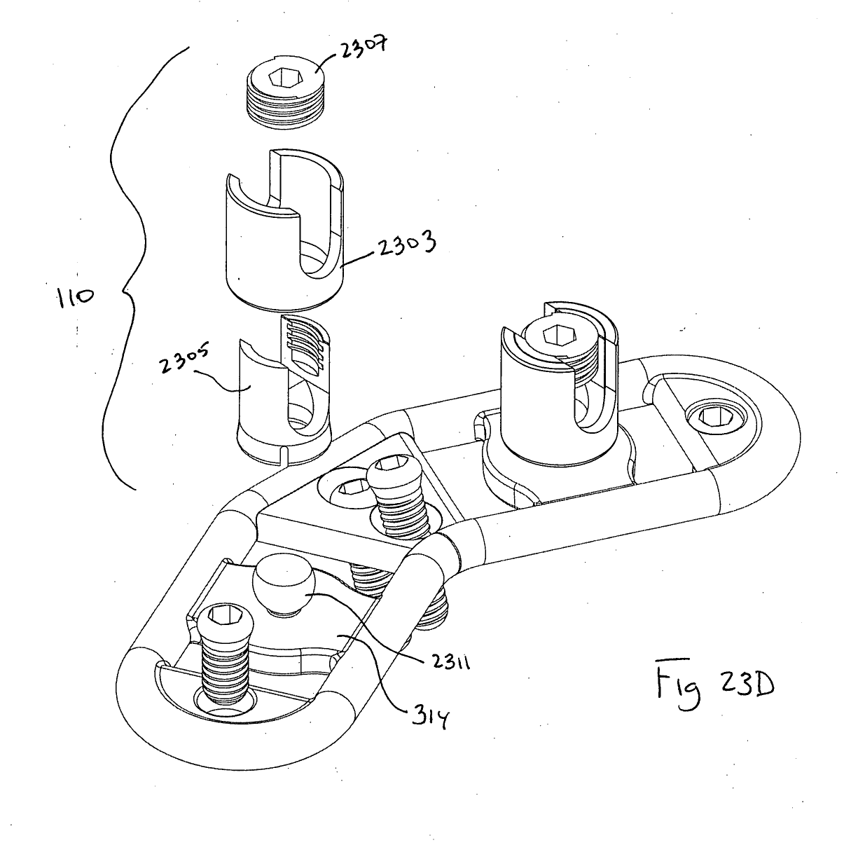

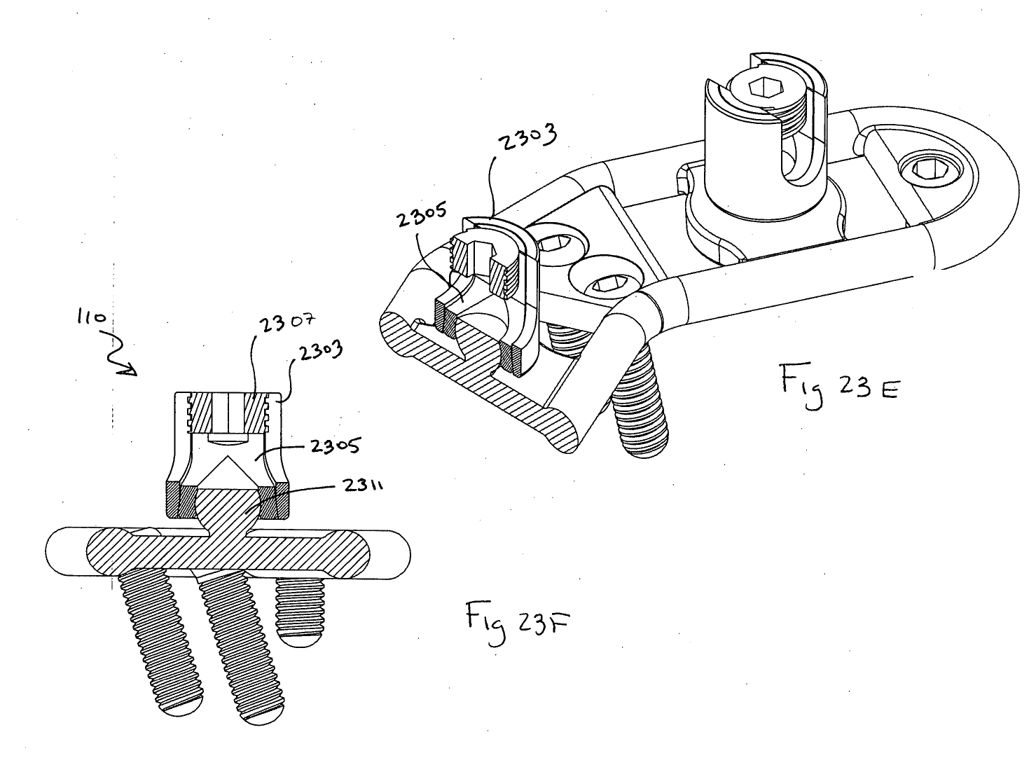

FIG. 21 shows a skull attachment 105 with an alternative embodiment of the rod fixation assembly 110. FIGS. 22A and 22B show perspective and top plan views of the skull attachment of FIG. 21. FIGS. 23A and 23B show cross-sectional views of the skull attachment of FIG. 21. This embodiment includes a cup-like outer member 4005 and a pair of locking members 4010 that can be positioned within the outer member 4005. When compressed, the locking members 4010 are adapted to lock onto rod 115 and upward protrusion 4015 located on an extension 314 of the skull attachment. When the rod fixation assembly 110 is moved into a locked position, it immobilizes the rod 115 relative to the skull attachment 105.

The rod fixation assembly 110 is moved into the locked position by inserting the rod 4005 onto a seat 4018 (FIG. 21) formed by the locking members 4010. The rod is pressed downward so as to press the locking members 4010 deep into the outer member 4005 such that the locking members 4010 tightly fits within the inner surface of the outer member 4005, as shown in FIGS. 23A and 23B. The locking members 4010 can lock within the outer member 4005 using an interference taper mechanism (such as a Morse taper). The two locking members 4010 are forced inward to immobilize the rod therebetween and to also squeeze the protrusion 4015 therebetween. In this way, the rod fixation assembly 110 serves to lock the rod 115 relative to attachment 105. Although a Morse taper locking mechanism provides a powerful immobilization, it may be loosened with only a modest backout of the locking members 4010 relative to the outer member 4005. This may be prevented by the addition of a ratchet locker, wedge locker, protrusion/indentation locker, or any other appropriate locking mechanism.

FIG. 23C shows a skull attachment 105 with an alternative embodiment of the rod fixation assembly 110. FIG. 23D shows the skull attachment 105 with the rod fixation assembly in an exploded state. Like the embodiments of the skull attachment of FIGS. 2 and 21, the skull attachment in FIGS. 23C-D is formed of a rod that defines a structural framework of the attachment.

This embodiment of the rod fixation assembly 110 includes a cup-like outer member 2303 and a cup-like inner member 2305 that fits concentrically within the outer member 2303. A locking nut 2307 mates with internal threads in the inner member 2305. The locking nut 2307 can be threaded into the inner member 2305 to immobilize a rod that is positioned within a slot in the outer member 2303. The outer member 2303 and inner member 2305 are adapted to lock onto a rod and also lock onto an upward protrusion 2311 (FIG. 23D)located on an extension 314 of the skull attachment. When the rod fixation assembly 110 is moved into a locked position, it immobilizes the rod 115 relative to the skull attachment 105.

With reference to the cross-sectional views of FIGS. 23E and 23F, in the assembled state, the outer member 2303 and inner member 2305 are positioned such that the protrusion 2311 is disposed within a seat located in the bottom region of the inner member 2305. The seat and the protrusion 2311 have complementary, rounded shapes such that the seat and the protrusion 2311 are arranged in a ball and socket manner. This permits the inner member 2305 and the outer member 2311 to be swiveled around the protrusion 2311 to adjust the orientation of a rod positioned in the outer member.

The rod fixation assembly 110 can be locked such that the inner and outer members and the rod are immobilized relative to the skull attachment 105. The rod fixation assembly 110 is moved into the locked position by inserting the rod into the slot in the outer member 2303 and then tightening the locking nut 2307 downward such that it compresses the rod against the upper end of the protrusion 2311. Advancement of the rod and the locking nut toward the protrusion causes the inner member 2305 to move upward while the outer member 2303 moves downward due to the respective shapes of the contact surfaces between the inner and outer members. The protrusion is thereby compressed by the inner and outer members to immobilize the rod fixation assembly on the protrusion.

Connectors

An inter-connector is used to affix the occipital attachment onto the cervical fasteners. While rods are most commonly employed, plates, combination rod/plate inter-connectors or any other inter-connection device may be alternatively used. In the following embodiments, rods inter-connectors are illustrated for simplicity but the current invention is not limited to them. It is understood that any other inter-connector device may be alternatively used to affix the skull attachment onto the cervical fasteners and that the following connectors may be similarly used with those inter-connector devices.

An occipito-cervical connector that enables rod 115 to be coupled to rod 120 is now described. The connector permits relative movement between the two rods in one or more planes. The system includes a locking mechanism that can be actuated to lock the relative positions of the rods. As mentioned, the rods can provide to connect an attachment(s) on the skull (such as the sub-occipital attachment) and an attachment(s) on the spine (such as spine screw(s)).

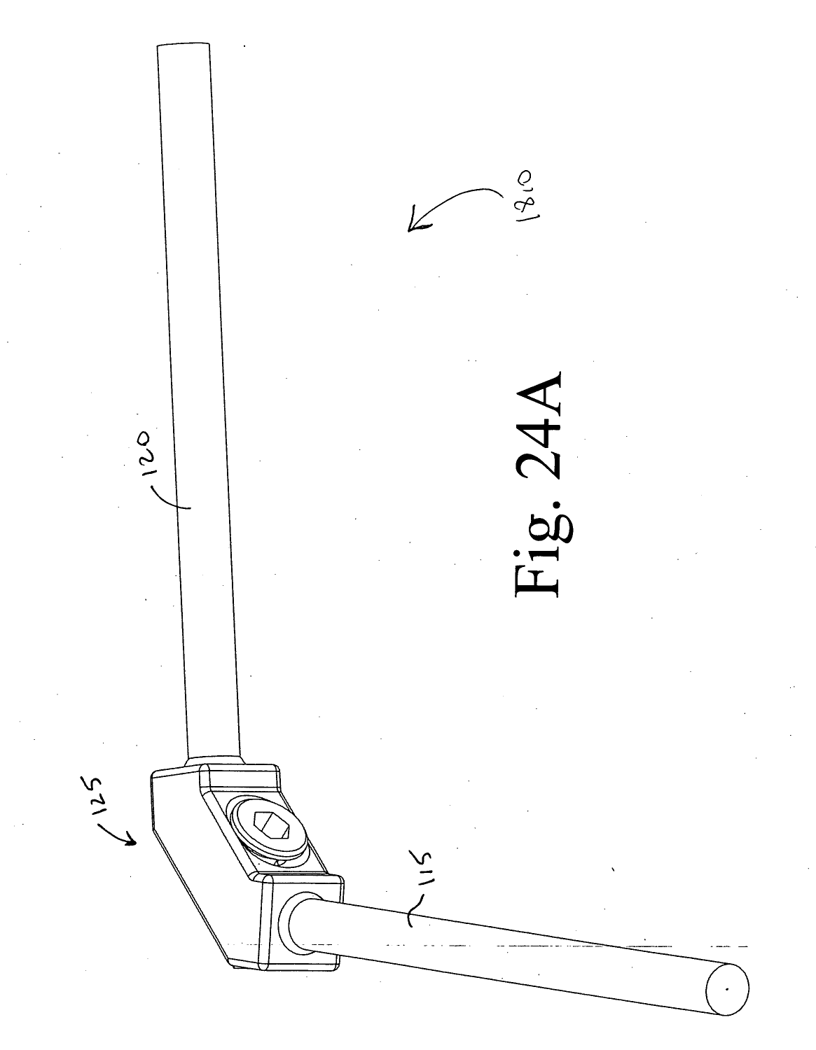

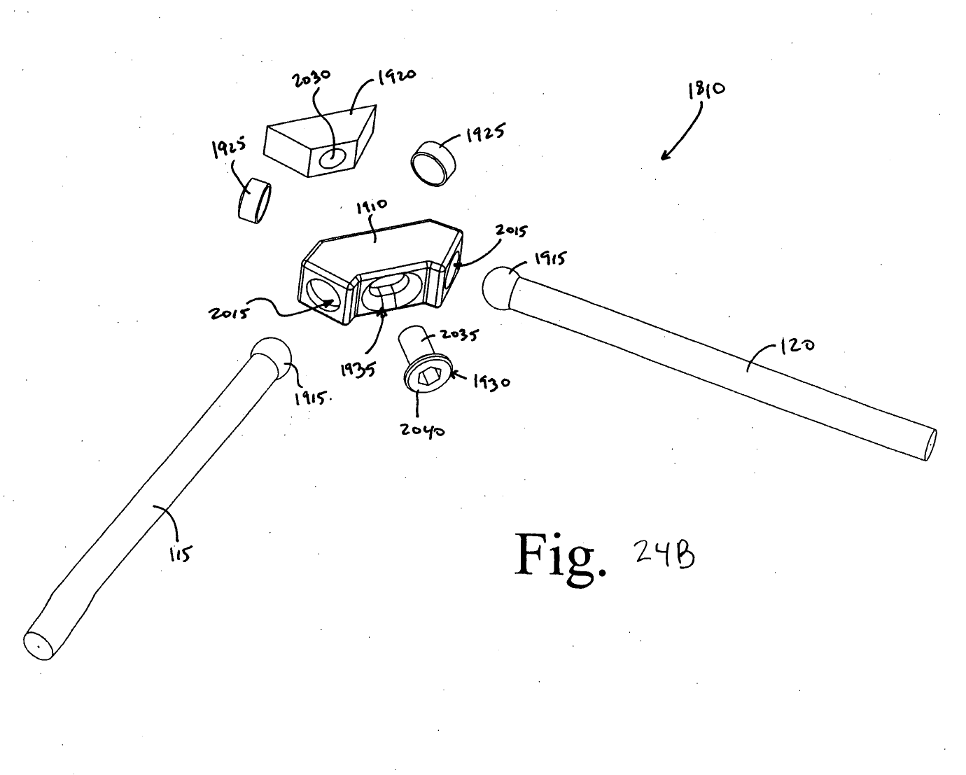

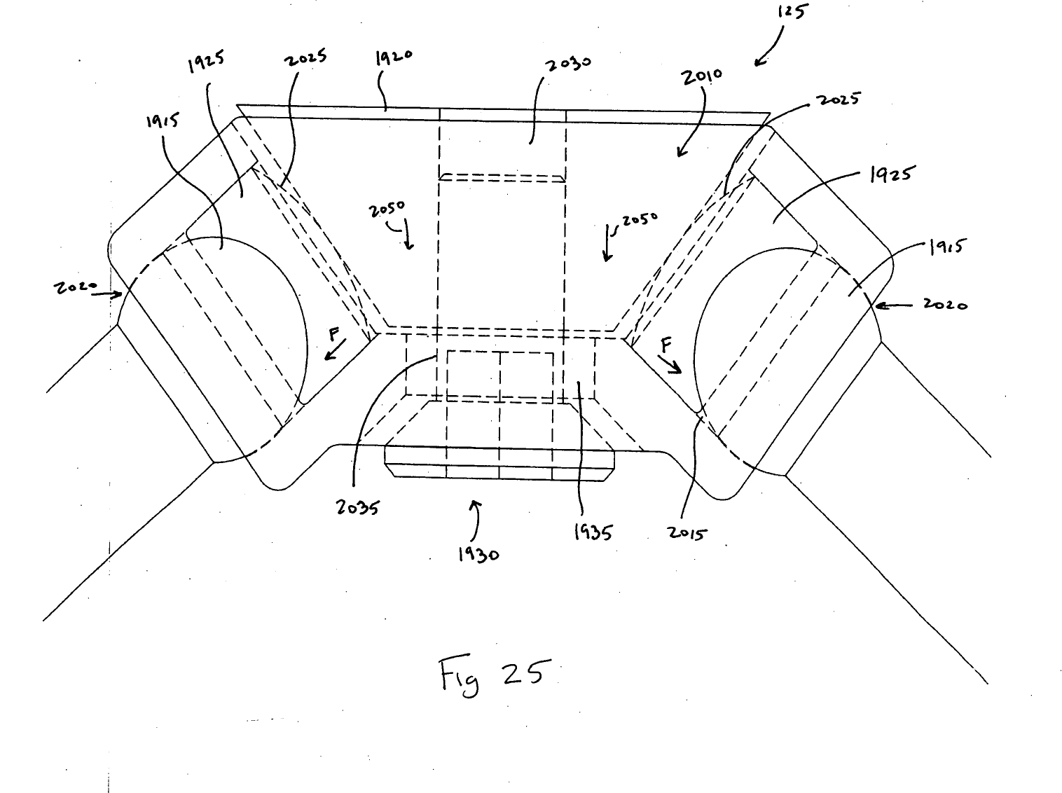

FIG. 24A shows a perspective view of a first embodiment of an occipito-cervical connection system 1810 that includes the connector 125, the rod 115, and the rod 120. The connector 125 permits the distal ends of rod 115 and rod 120 to mate with the skull attachment(s) and the cervical attachment(s), respectively, without having to contour the rods. Advantageously, the connector 125 connects rod 115 to rod 120 in such a way that the relative position of the two rods can be adjusted in one or more planes.

FIG. 24B shows an exploded view of the connector 125 of FIG. 24A, which includes a main housing 1910 that couples to a head 1915 located on a proximal end of each of the rods 115 and 120. The connector 1910 further includes a locking member 1920 that can be positioned at least partially within the housing 1910. The locking member 1920 preferably interacts with a pair of caps 1925 that each interact with a respective head 1915 of rods 115 and 120. An actuator member 1930 adjustably engages the housing 1910 (via a channel 1935 in the housing 1910) and the locking member 1920 (via a bore 2030 in the locking member 1910) to lock the positions of rods 115 and 120 after the desired orientation is achieved.

FIG. 25 shows a cross-sectional view of the connector 125 in an assembled state and coupled to rod 115 and rod 120. In the illustrated embodiment, the housing 1910 has a central portion with an internal, wedge-shaped cavity 2010 that receives the locking member 1910. The locking member 1910 has a complementary wedge-shape that fits within the cavity 2010. Lateral portions of the housing 1910 each include a bore 2015 that forms an opening 2020 in the housing 1910.

The spherical head 1915 of each rod is sized to fit within a corresponding bore 2015. The opening 2020 for each bore is smaller than the maximum transverse dimension of the corresponding head 1915 but greater than the transverse dimension of the rod attached to the head. Thus, the head 1915 cannot slide through the opening 2020 but the rod can transverse the opening. When the head of a rod is fully seated within bore 2015, opening 2020 prevents head 1915 from sliding out of housing 1910.

With reference still to FIG. 25, a cap 1925 sits atop the head 1915 of each rod. Each cap 1925 has an abutment surface 2025 that extends at least partially into the cavity 2010. The locking element 1920 sits within the cavity 2010 such that a locking caps 1925 is interposed between the locking element 1920 and a respective head 1915 of a rod. Alternately, the cap(s) 1925 may be eliminated altogether and the side arms of the housing 1910 made sufficiently shallow so that a portion of the heads 1915 sits within the cavity 2010. In this way, the locking element 1920 makes direct contact with the heads 1915. The locking element 1920 includes an internally-threaded, mid-line bore 2030 that aligns with the channel 1935 of the housing 1910 when the locking element 1920 is positioned within the cavity 2010.

In the embodiment shown in FIG. 25, the actuator member 1930 comprises a screw having a threaded post 2035 and a screw head 2040. The threaded post 2035 is sized and shaped to engage the threaded bore 2030 in the locking element 1920. The head 2040 of the actuator member 1930 can include a coupling structure, such as, for example, a hexagonal cavity, that is configured to receive a complimentary driver, such as, for example, an Allen-style wrench, for rotating and driving the actuator member 1930 into the locking element 1920.

The mechanism for adjusting and locking the rods using the connector 125 is now described. After the distal end of each rod is fixed to either the skull and/or cervical attachment(s), head 2040 of actuator member 1930 is engaged. Rotation of the actuator member 1930 draws the locking element 1920 deeper into the wedge-shaped cavity 2010 in the housing 1910, as exhibited by the arrows 2050 in FIG. 25. As the locking element 1920 advances deeper into the cavity 2010, it contacts the curvilinear abutment surface 2025 of each cap 1925 and drives the caps 1925 further into the bores 2015. The caps 1925 exert a force F on the heads 1915 of the rods. The force F causes the spherical heads 1915 to sit tightly within the respective bores 2015 and immobilizes the spherical head 1915 of each rod within the respective bore in the housing 1920. In this way, the position of the rods 115 and 120 are locked relative to one another. The heads of the rods and the abutment surface of the caps may be further textured or ridged in order to increase the frictional contact between them and thereby increase the holding power of the device. The locking strength may be further augmented by using caps that conform and lock onto the spherical heads of the rods or by using a separate member that provides that function. These cap and the additional member designs are well known in the art and are illustrated in U.S. Pat. Nos. 6,623,485; 6,565,565; 5,733,285; 5,176,680 and numerous others.

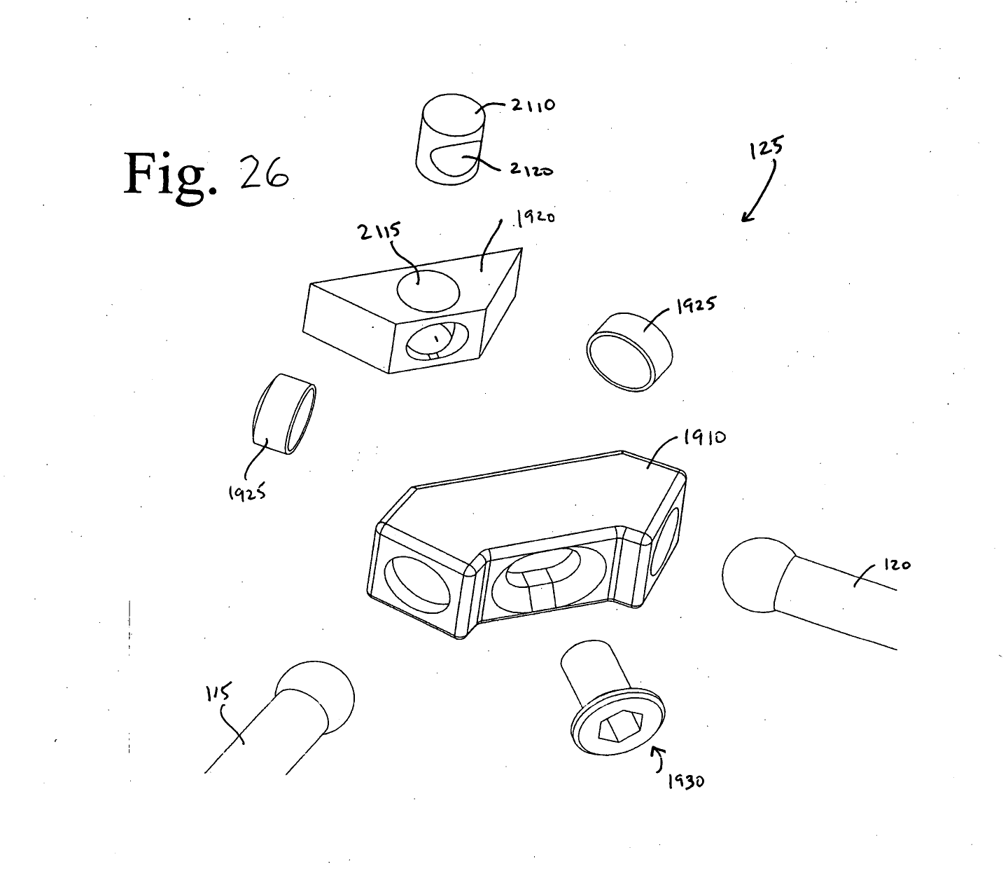

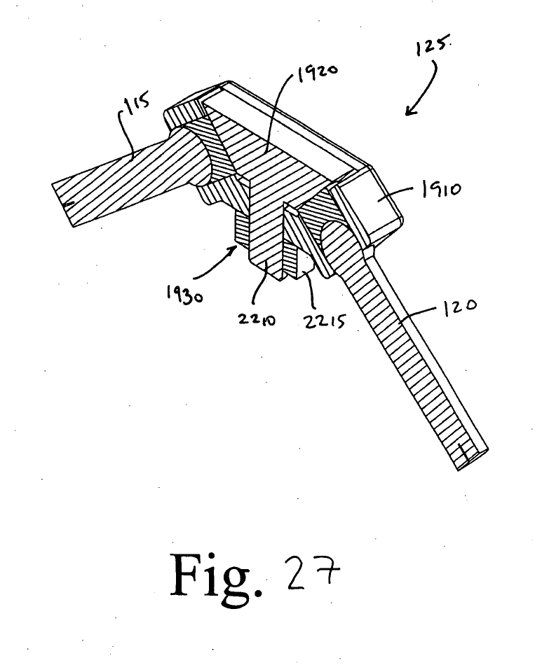

FIG. 26 shows another embodiment of the connector 125 in which a barrel nut 2110 is positioned inside a bore 2115 within the locking member 1920. The barrel nut 2110 has a threaded bore 2120 that is sized to receive a complementary-threaded post 2035 of the actuator member 1930 for tightening the locking member 1910 within the housing 1910.

It should be appreciated that various other mechanisms can be used to lock the position of the rods. For example, FIG. 27 shows an embodiment of the connector 125 wherein the locking member 1920 has a threaded post 2210 that protrudes out of the housing 1910. The actuator is a threaded nut 2215 that engages the post 2210. As nut 2215 is tightened, it pulls locking member 1920 deeper into the cavity of housing 1910 and locks rods 115 and 120 in position.

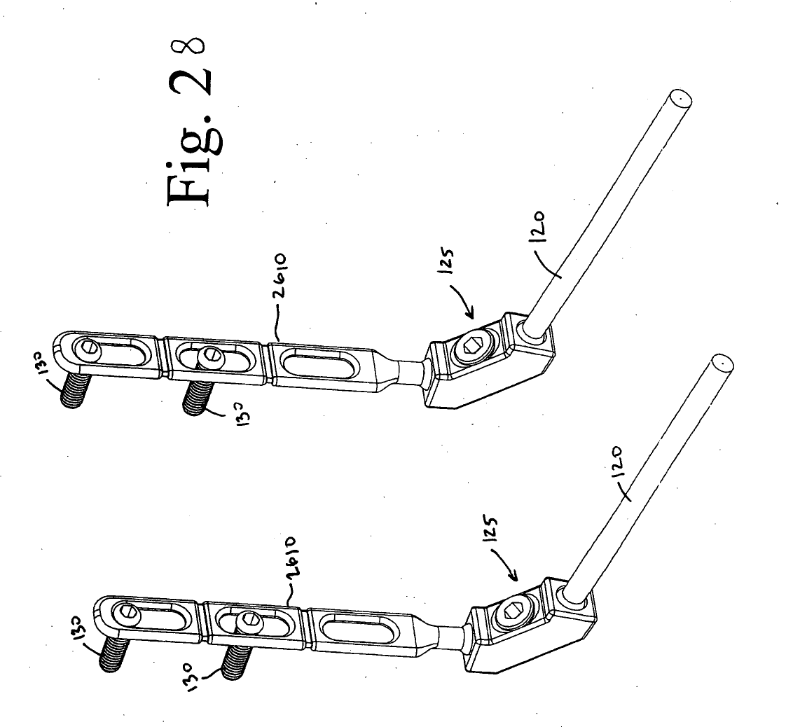

FIG. 28 shows another embodiment wherein rod 115 is replaced with an elongate interconnector comprised of an elongated plate member 2610 and attached to rod 120 via any of the previous embodiments of the connectors 125. Member 2610 is an elongated inter-connector with a plate-like configuration that includes one or more apertures. The apertures are sized to receive bone fasteners, such as screws 130, which attach member 2610 to the skull. Other types of fasteners, such as buttons, can be alternatively used to attach the plate member 2610 to the skull. The embodiment shown in FIG. 28 may be attached directly onto the skull or it may be attached onto a skull attachment that is already affixed onto the skull. In an additional embodiment, both rods 115 and 120 may be replaced by elongated members 2610.

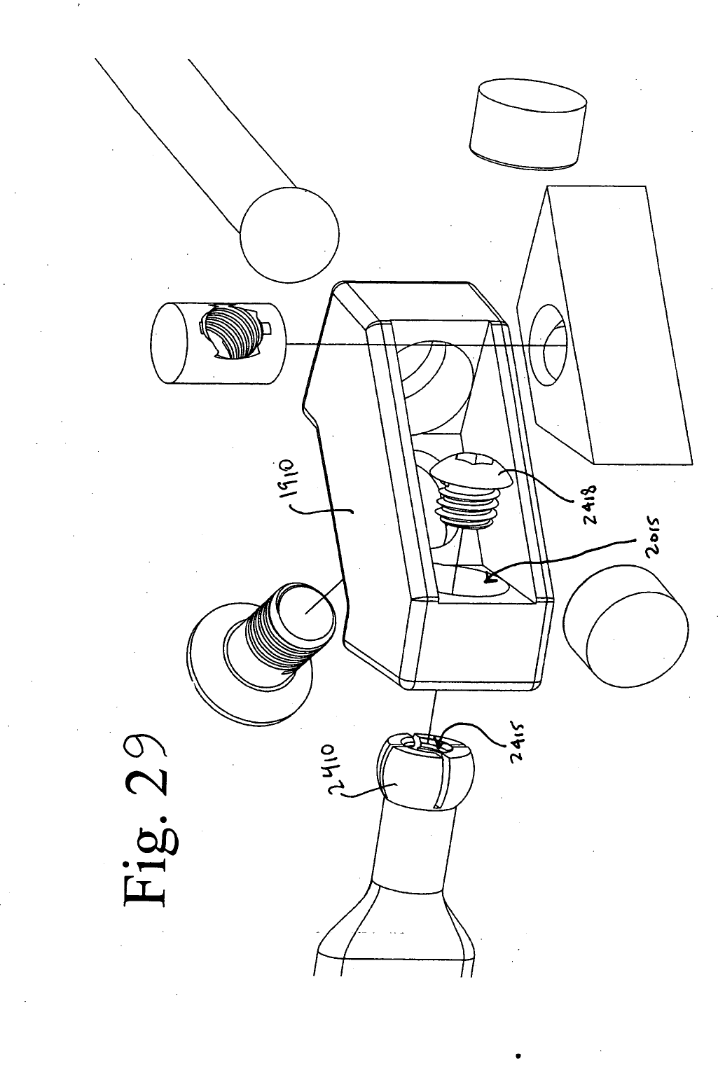

FIG. 29 shows yet another embodiment of the connector wherein at least one of the rods utilizes a collapsible head 2410. The head 2410 has a threaded bore 2415 that receives a screw 2418. The collapsible head 2410 can be pushed into bore 2015 of housing 1910. Once inside bore 2015, the threaded screw 2418 mates with head 2410 and renders the head un-collapsible so that head 2410 is retained within housing 1910.

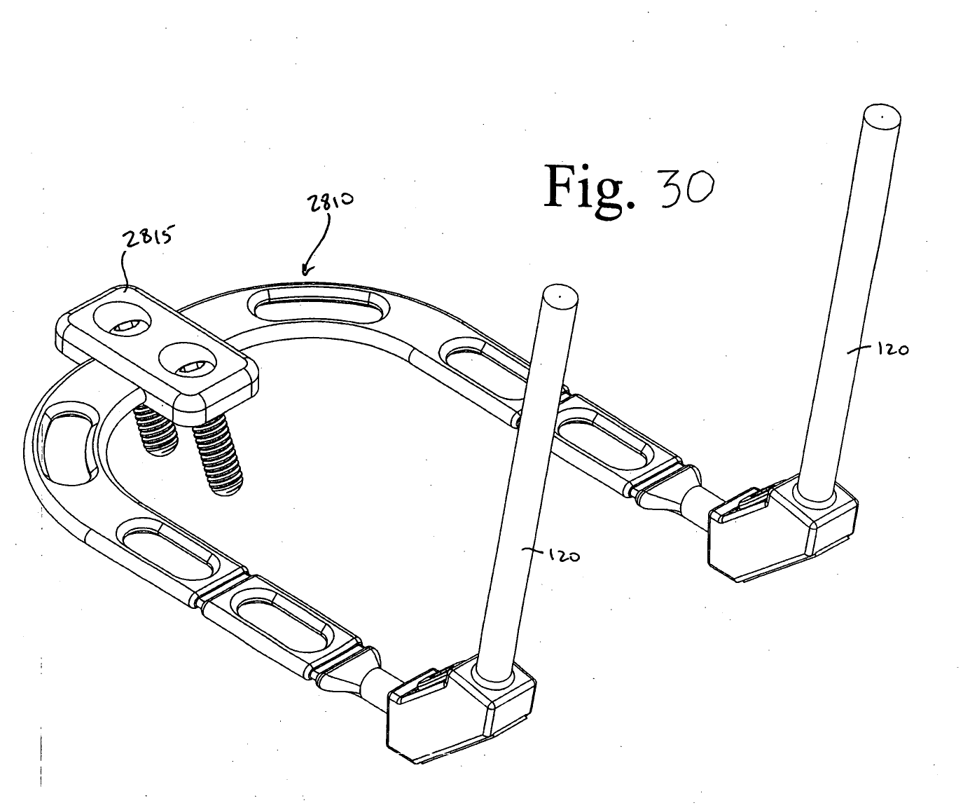

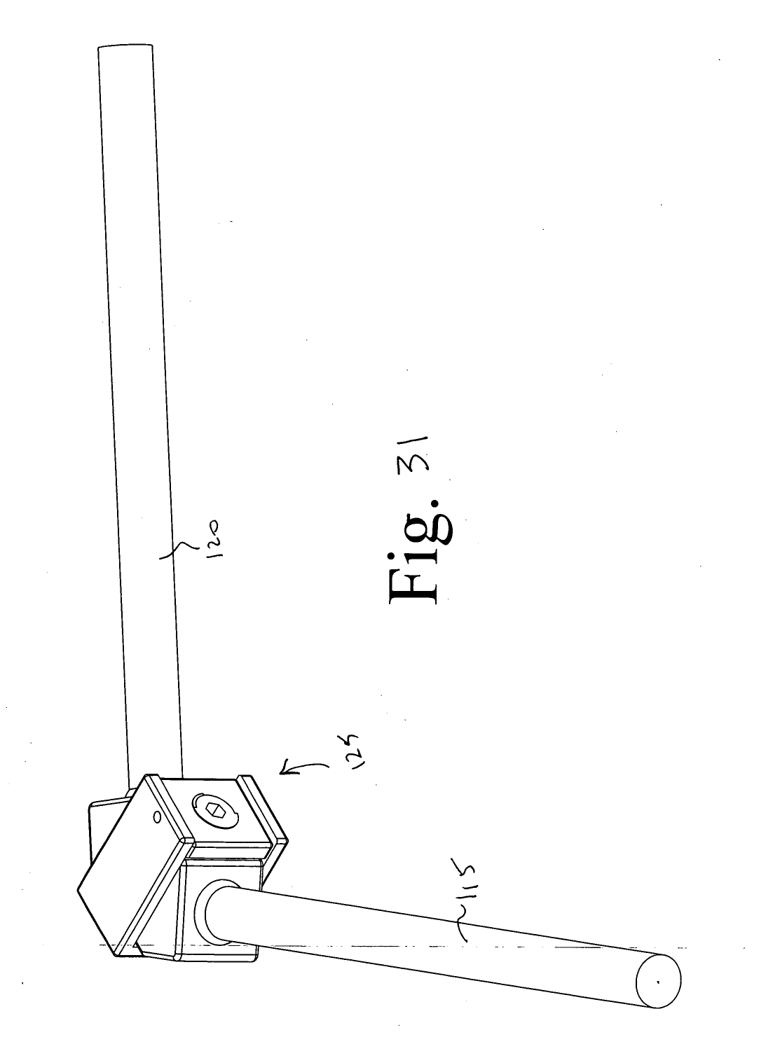

FIG. 30 shows yet another embodiment wherein rods 115 and the skull attachment 105 are collectively replaced by a skull connector comprising a “U”-shaped plate 2810 having a central member 2815. The central member 2815 can differ in thickness from the plate 2810 or it can be of similar or same thickness. The central member 2815 has boreholes for receiving bone screws, wherein the boreholes are preferably adapted to permit poly-axial screw placement and the borehole trajectory is preferably non-perpendicular relative to the top surface of the central member. The “U”-shaped plate 2810 has a plurality of apertures that can receive additional bone fasteners, such as screws or buttons, to provide additional points of fixation onto the underlying bone. In any of the embodiments described herein, other fasteners, such as cables and the like, can be alternatively used.

Method of Use

In use, the skull attachment 105 is placed at the posterior aspect of the skull and centered over the midline of the underlying bone. The device is preferably, but not necessarily anchored onto the sub-occipital skull.

The skull attachment 105 attaches to the sub-occipital bone using the bone screws 130 or other fasteners (shown in FIG. 1), which are placed through the boreholes within central body 305 and into the underlying bone. The bone screws follow the angled trajectory of the bore holes and attach to the underlying bone in a trajectory that is preferably not perpendicular to the bone surface. As mentioned, additional bone screw, button attachments or other fasteners (cables, etc) may be placed through the remaining boreholes to provide additional fixation. The screws are preferably placed in a cross-screw configuration so as to optimize the fixation of the skull attachment onto bone.

Each rod is cut to appropriate length. On each side of the skull attachment 105, a rod 115 is attached to attachment 105 via the rod fixation assembly 110 (shown in FIG. 1). The cervical rods 120 are then attached to at least a portion of the cervical spine 2115 using one or more cervical attachments in a well-known manner. The cervical rod 120 is preferably locked into the cervical attachment(s) first. Subsequently, rod 115 is locked into the rod fixation assembly 110 of the skull attachment 105.

Connector 125 couples rod 115 to rod 120. After the rods have been attached as described above, the connector between them is locked. While the preferred order of lock deployment is illustrated, the locks may be deployed in any order that the surgeon prefers. Once all connection points are tight, the construct is rigid.

Advantageously, the connector 125 permits movement of rods 115 and 120 relative to one another in one or more planes. The relative positions and orientations of the rods can be adjusted and then locked in the desired orientation. The connector 125 does away with the current surgical practice of contouring the rod(s) that inter-connect the skull attachment and cervical attachment(s).

### Additional Embodiments of the Connector

FIG. 31 shows a perspective view of another embodiment of an occipito-cervical connection system that includes the connector 125, the rod 115, and the rod 120. As mentioned, the connector 125 attaches rod 115 to rod 120 in such a way that the orientation between the two rods can be adjusted in one or more planes.

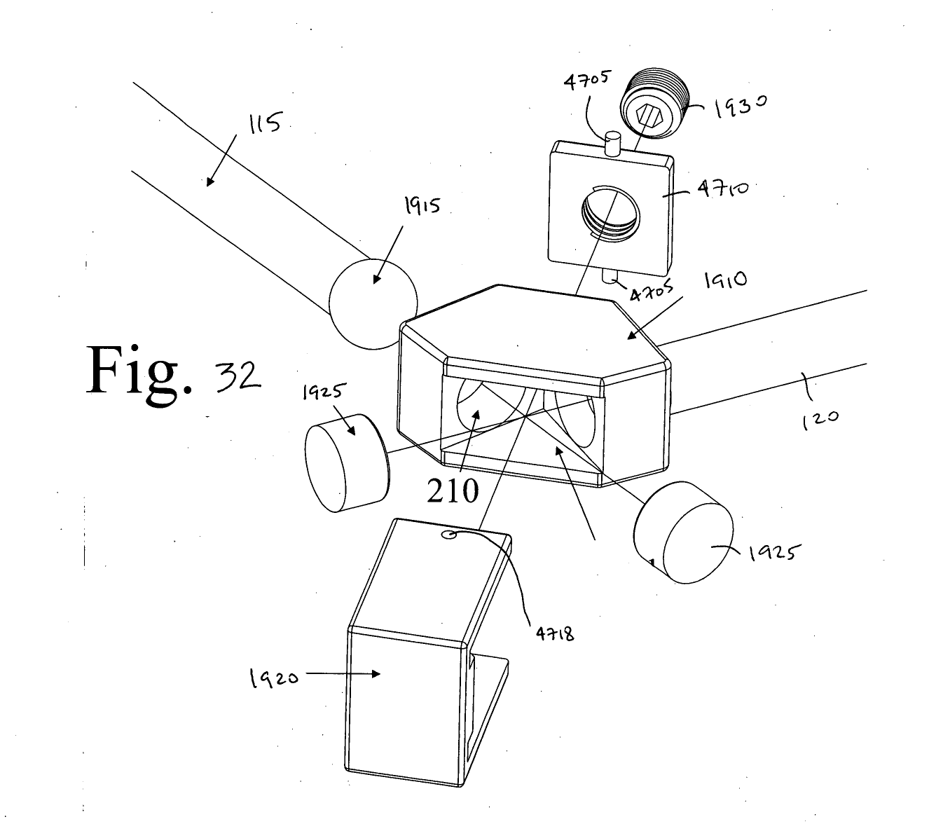

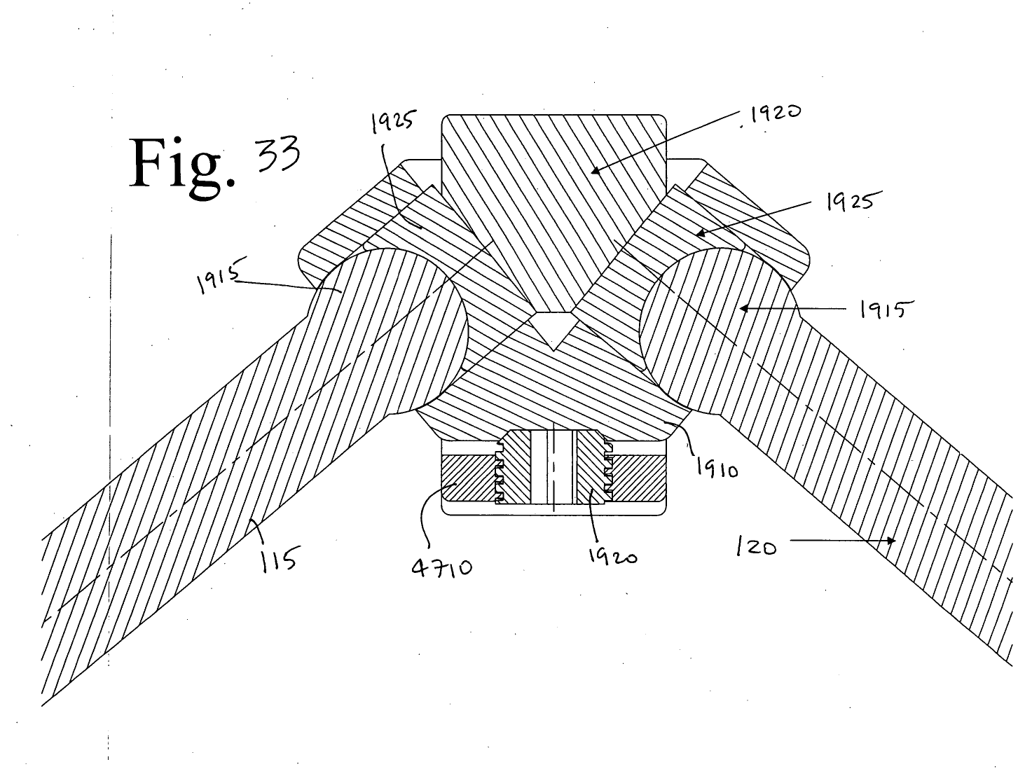

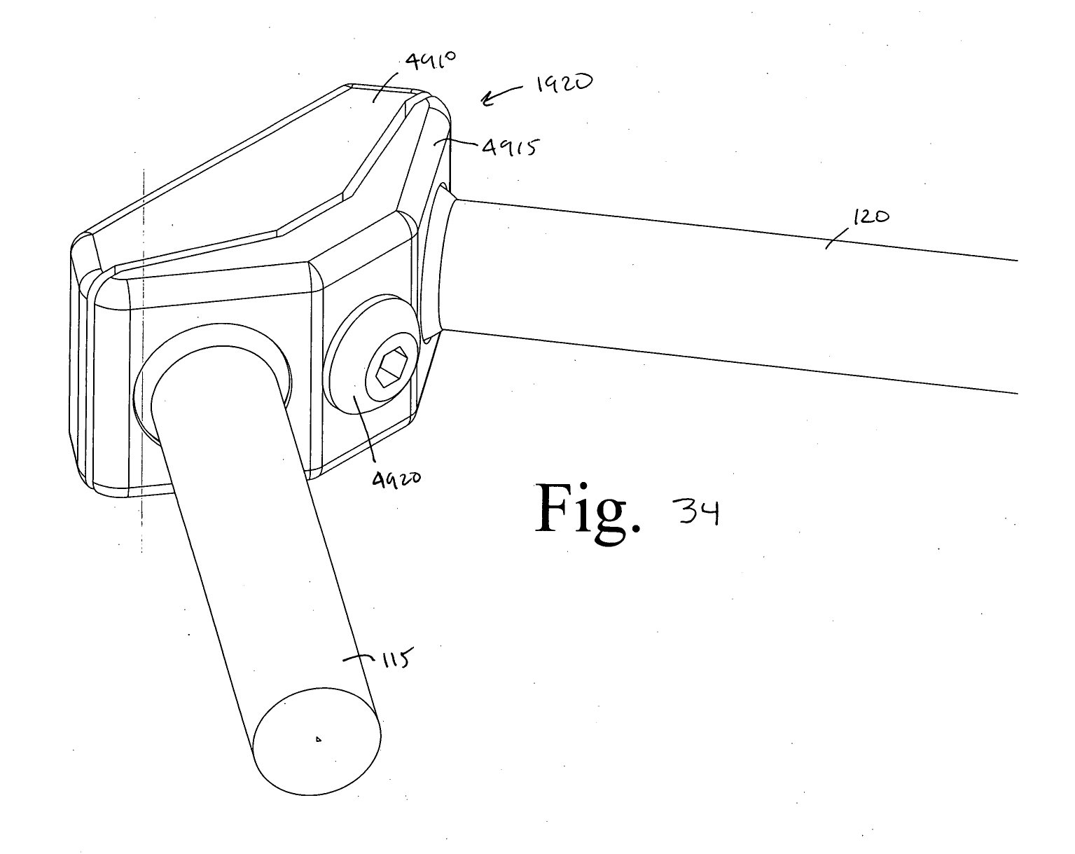

FIG. 32 shows an exploded view of the connector in FIG. 31, which includes a main housing 1910 that couples to a head 1915 located on a proximal end of each of the rods 115 and 120. The connector 1910 further includes a locking member 1920 that can be positioned at least partially within the housing 1910. The locking member 1920 interacts with a pair of caps 1925 that each interact with the respective head 1915 of the rods 115 and 120. An actuator member 1930 engages member 4710 to lock rods 115 and 120, as described in more detail below. Member 4710 has a pair of protrusions 4705 that mate with holes 4718 on the locking member 1920. FIG. 33 shows a cross-sectional view of the connector of FIG. 32. In use, the actuator member 1930 (comprised of a locking nut) is tightened into a threaded bore in member 4710 which causes member 1920 to be pulled deeper into the housing 1910 by virtue of the attachment between the member 4710 and the locking member 1920. As actuator member 1930 is tightened, locking member 1920 is wedge tightly against the caps 1925 and lock the heads 1915 of the rods 110 in a desired orientation within the housing 1910.

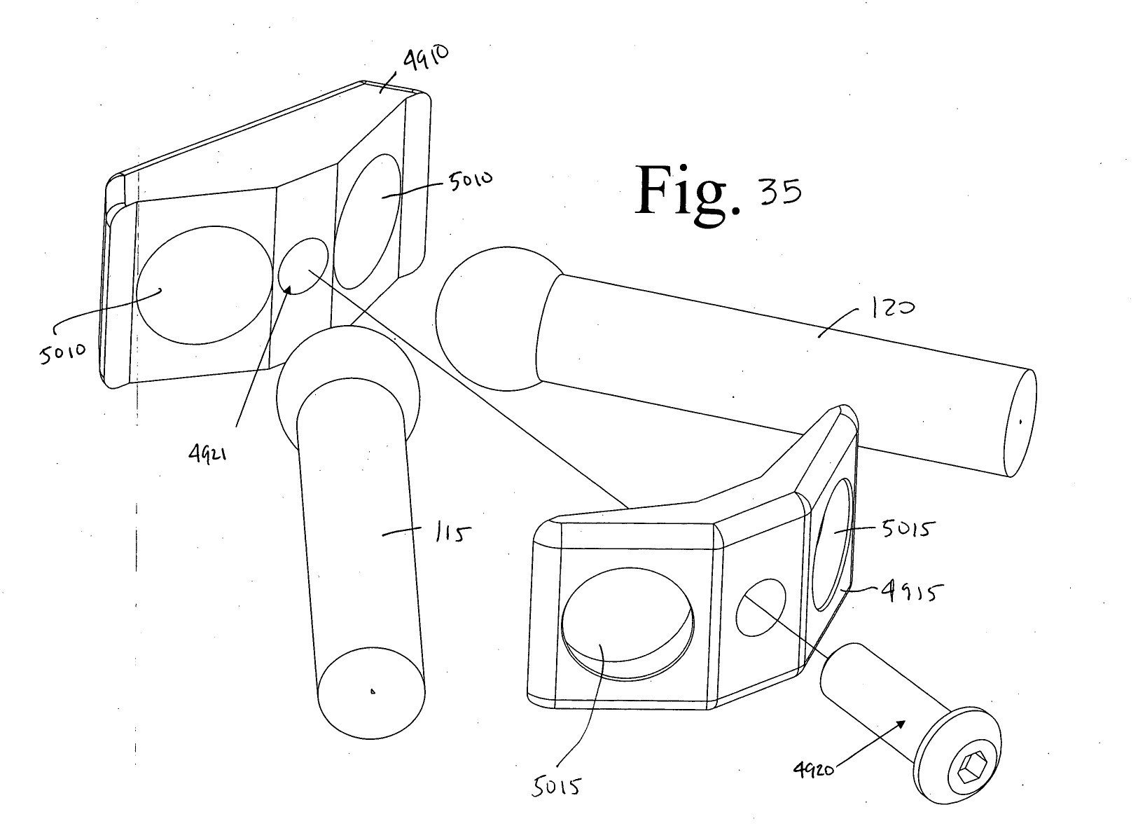

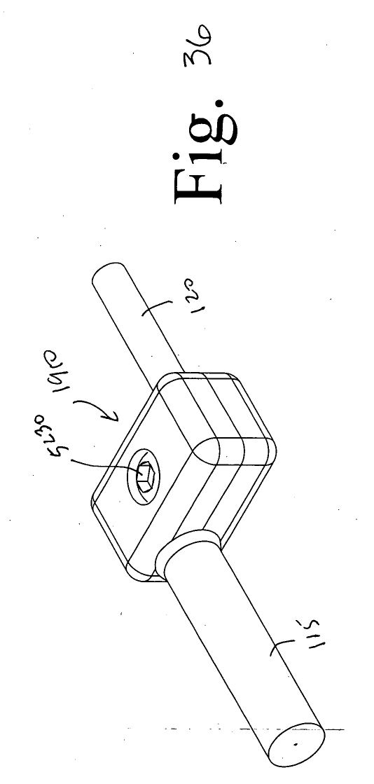

FIGS. 34 and 35 show an additional embodiment of the connector in an assembled state and an exploded state, respectively. The housing 1910 includes a rear member 4910 and a front member 4915 (FIG. 35) that are removably attached to one another with a locking screw 4920 that mates with a bore 4921 in the rear member 4910. As best shown in FIG. 35, the rear member 4910 has a pair of contoured seats 5010 that are sized and shaped to receive the head 1915 of rods 115 and 120. The front member 4915 has a pair of holes 5015 through which the rods 115 and 120 can be positioned. The holes 5015 are large enough to receive the rods but they're smaller than the diameter of heads 1915. When the device is assembled as in FIG. 34, the locking screw 4920 is initially loose such that the heads of the rods can be rotated within the seats 5010. After the rods are placed in the desired orientation, the locking screw 4920 is then tightened causing head 1915 of each rod to be compressed between the front member 4915 and the rear member 4910. In this manner, the rods are immobilized and their relative orientation is fixed.

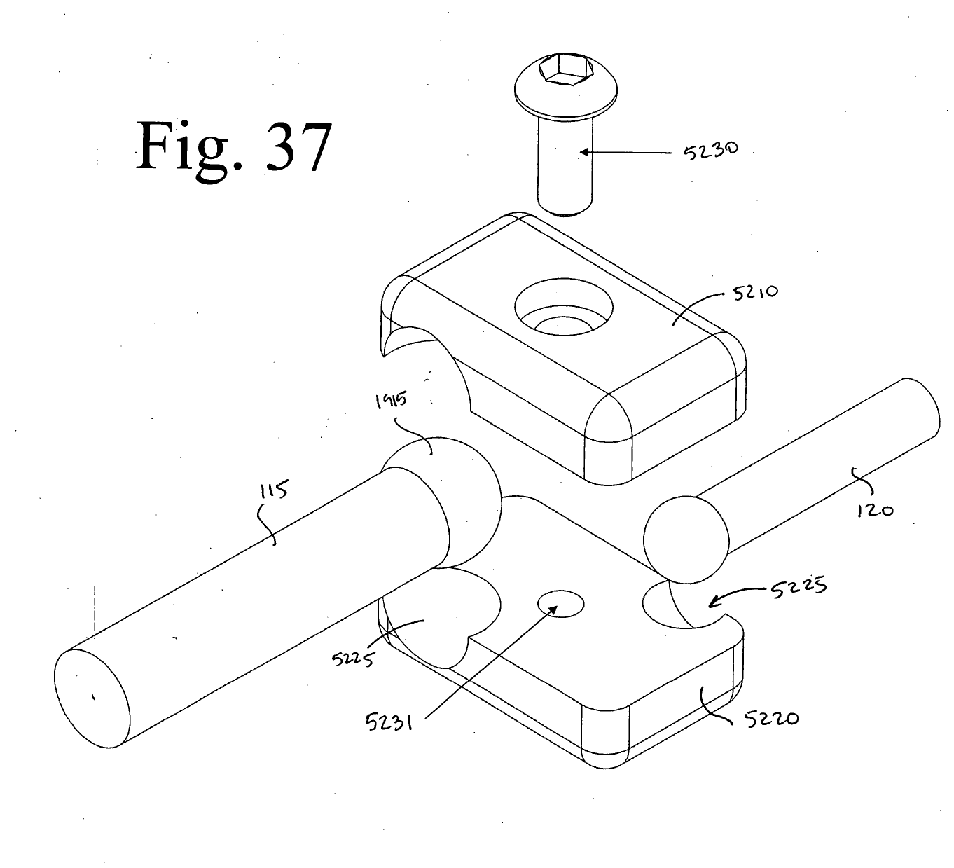

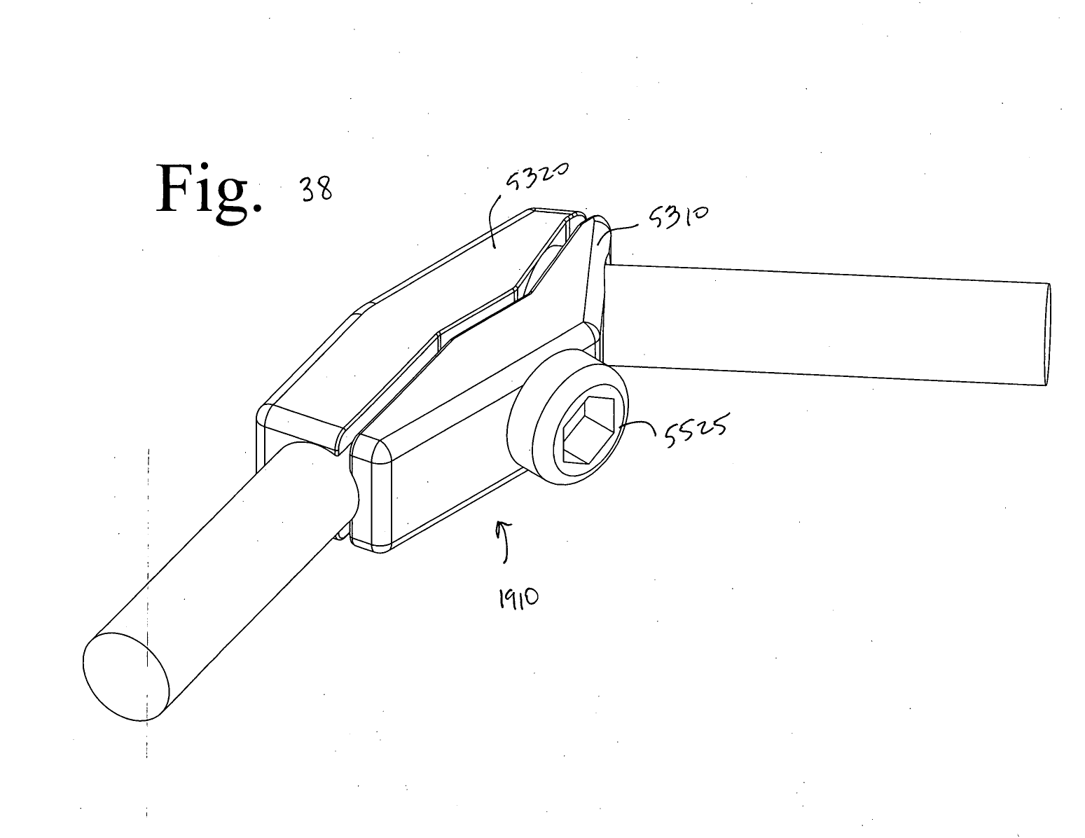

FIGS. 36 and 37 show another embodiment of the connector in an assembled and exploded state. In this embodiment, the rods 115 and 120 are offset relative to one another such that they can be aligned along offset axis. The connector includes a housing 1910 formed of a front member 5210 and a rear member 5220. The front member and rear member collectively form a pair of rounded seats 5225 in which heads 1915 of rods 120 and 115 can be positioned. After the rods are placed in the desired orientation, locking screw 5230 is tightened into a borehole 5231 so as to compress heads 1915 between the front member 5210 and the rear member 5220 and lock the rods in place.

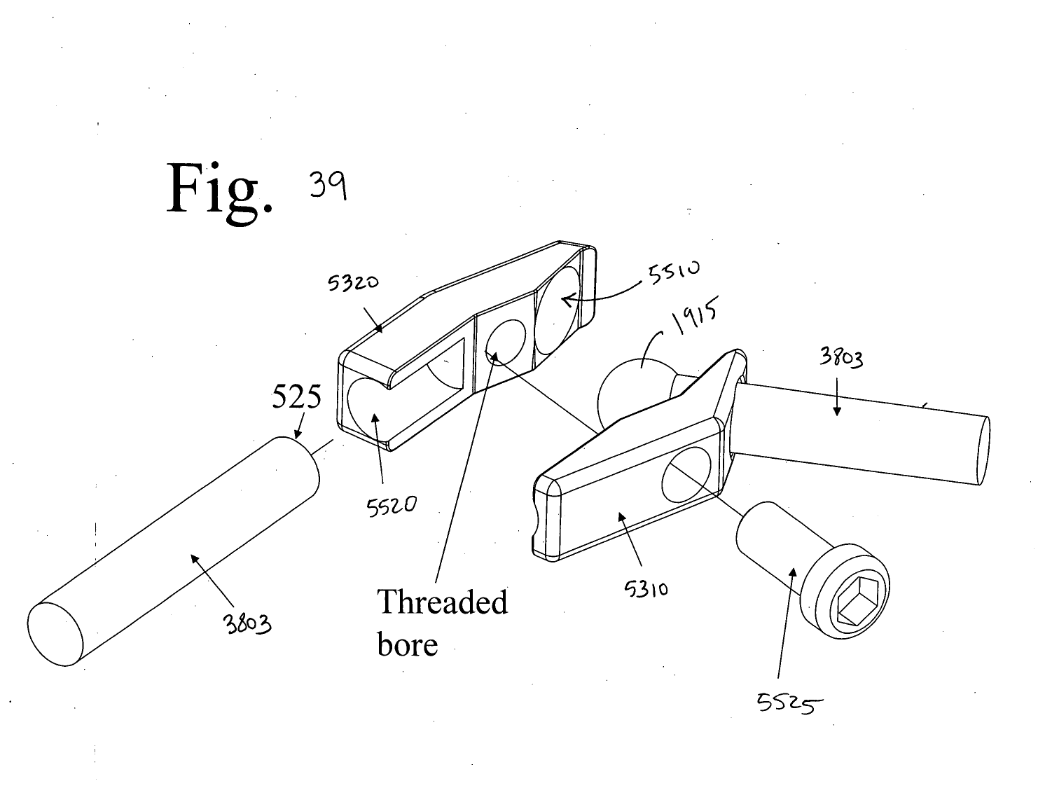

An additional connector embodiment is shown in FIGS. 38 and 39. In this embodiment, only one rod, 3802, has spherical head 1915, while rod 3803 is a conventional rod without head 1915. A housing 1910 is formed of a front member 5310 and a rear member 5320. The front and rear members collectively form a first seat 5510 that is adapted to receive the spherical head 1915 of rod 3802. The front and rear members also define a second seat 5520 that is sized and shaped to receive the end of the other rod. Rod 3802 can be freely rotated about the center of head 1915 until the locking screw 5525 is deployed. In use, rod 3803 is inserted into seat 5520 and rod 3802 is then moved into the desired orientation relative to rod 3803. Lock screw 5525 is tightened, rendering the device rigid and immobilizing rod 3802 relative to rod 3803.

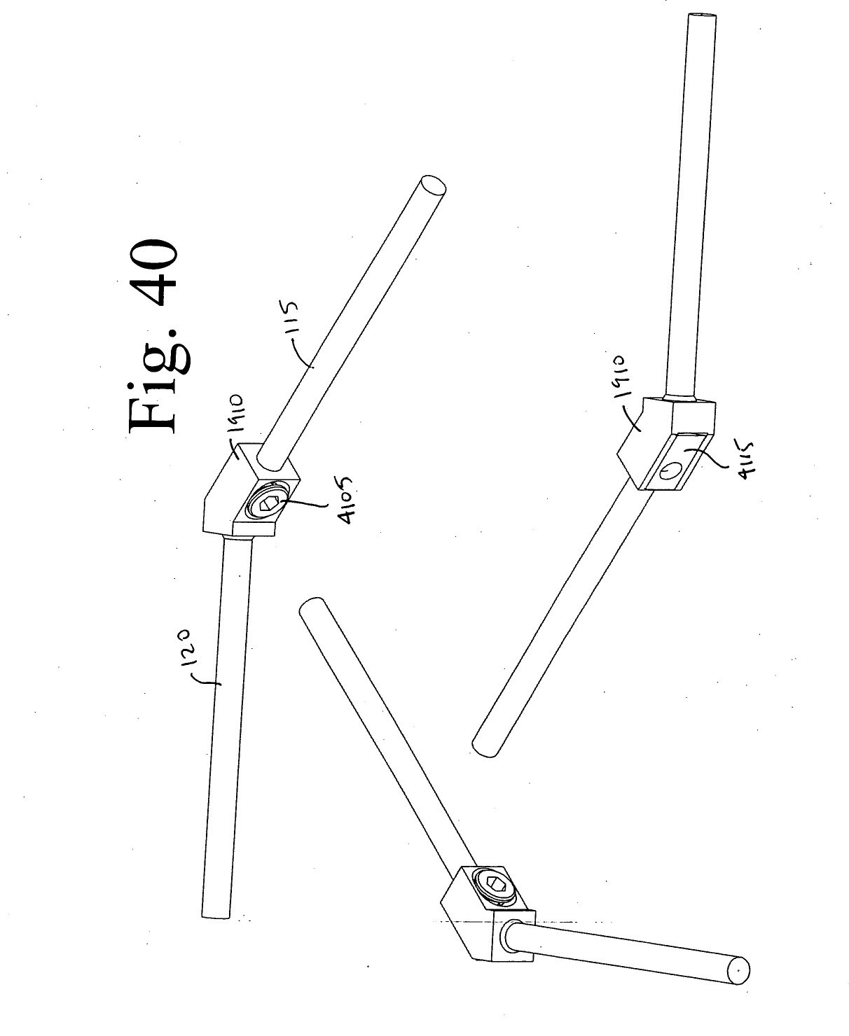

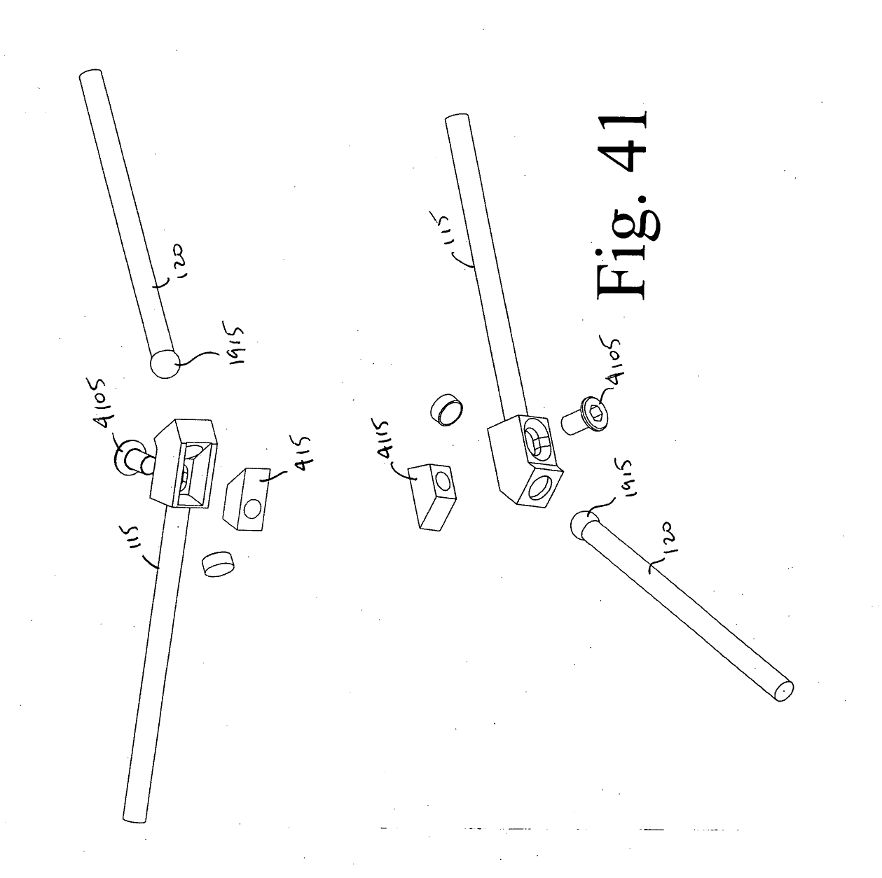

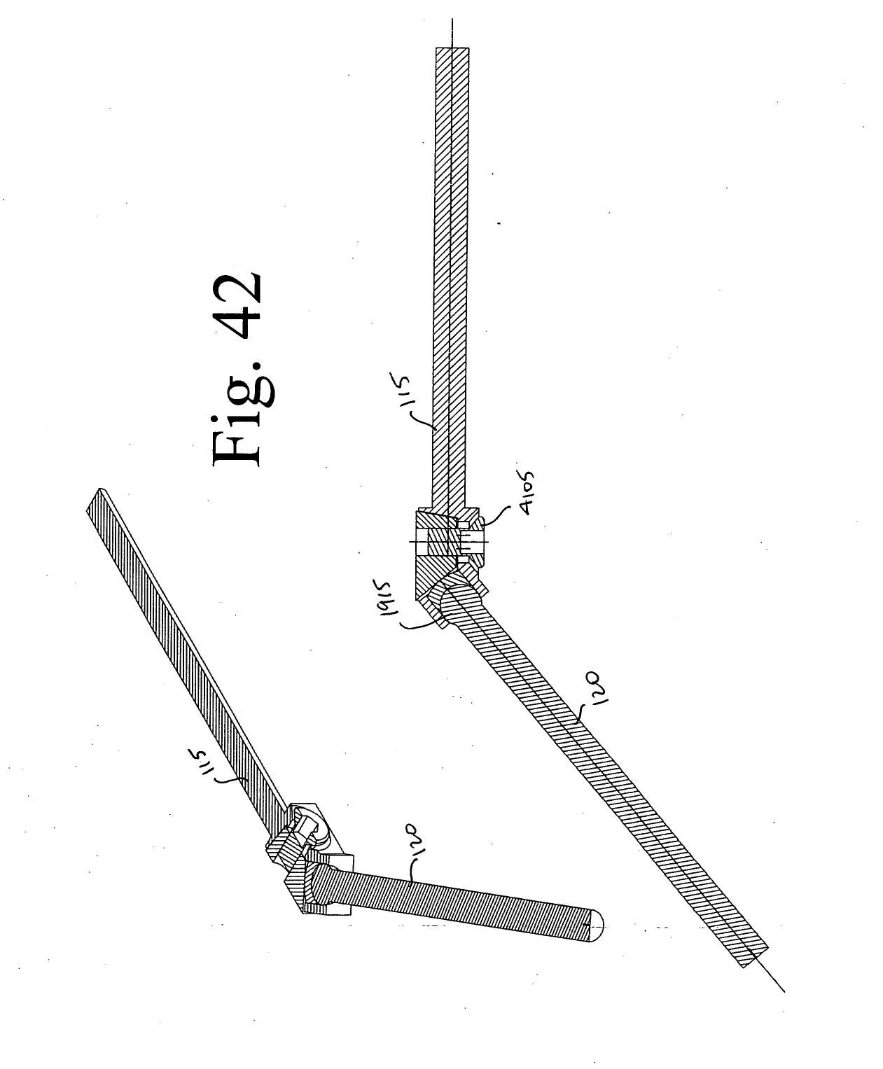

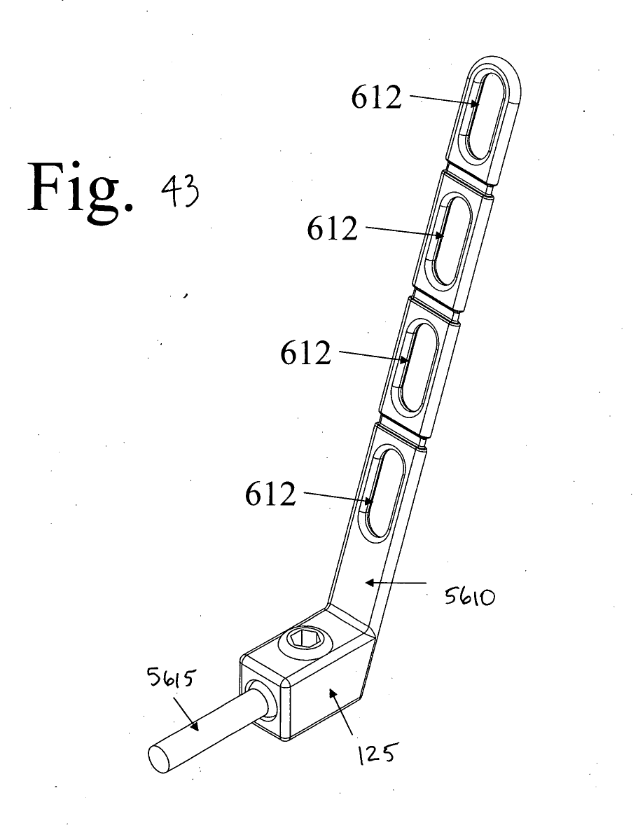

FIGS. 40-42 illustrate an additional connector embodiment. This connector includes a first rod 115 that is integrally attached to a housing 1910. A second rod 120 with a head 1915 can be secured within the housing by tightening a locking screw 4105, which interfaces with a locking member 4115. By tightening the locking screw, the locking member 4115 wedges tightly into the housing 1910 to compress the head 1915 in a fixed orientation in the housing 1910. Prior to tightening the locking screw, the head can be rotated such that the rod 120 is moveable in one or more planes.

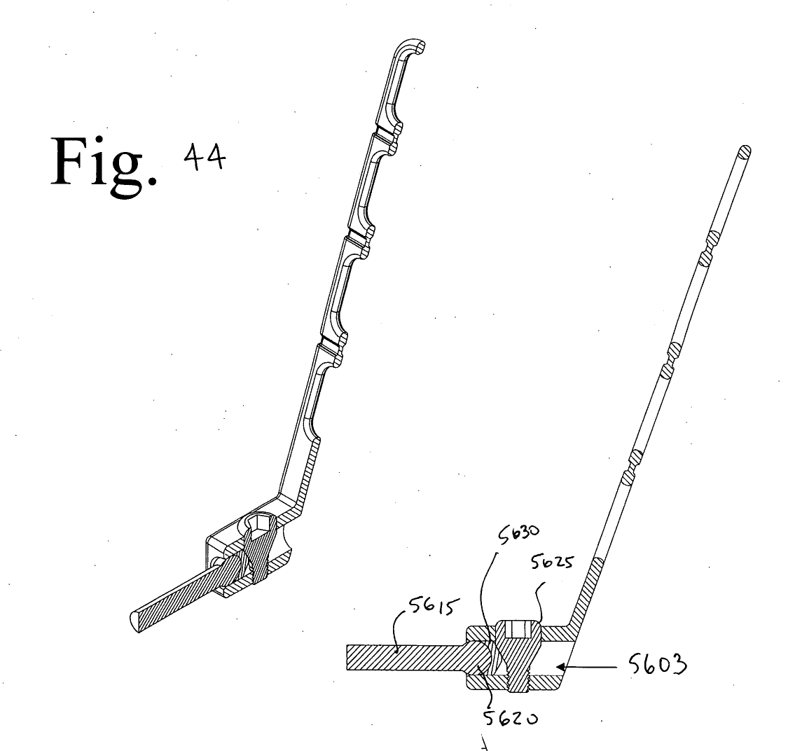

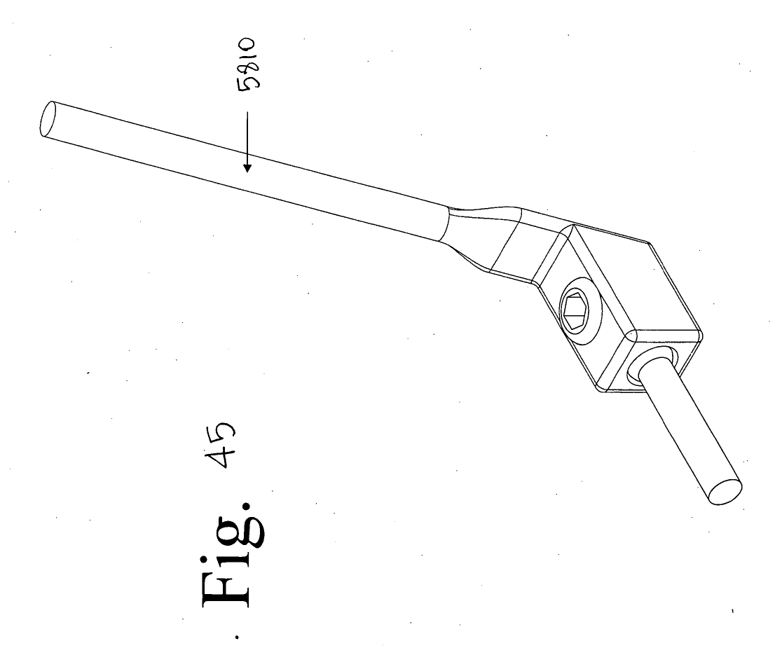

FIG. 43 shows another embodiment. An elongated plate member 5610 with apertures 612 is rigidly and integrally affixed to the connector. A rod 5615 is movably attached to the connector in a manner that permits the orientation of the rod 5615 to be adjusted. FIG. 44 shows perspective and side cross-sectional views of the connector of FIG. 43. The rod 5615 can be inserted into the connector 125 via the back end of bore 5603. The front end of bore 5603 is smaller that the diameter of the spherical head 5620 of rod 5615. Head 5620 sits within the connector 125 and rod 5615 is freely movable until the locking screw is deployed. Locking screw 5625 has a wedge-shaped head that abuts a wedge member 5630 in the housing. As the screw 5625 is tightened, the wedge member 5630 presses against the head 5620 of the rod 5615 and immobilizes the rod in place. The plate member 5610 is elongated and includes one or more apertures that are sized to bone fasteners, such as screws 130, for attaching the plate members 5610 to the skull. Other types of attachment devices, such as buttons, can be used to attach the plate members 5610 to the skull. In another embodiment (FIG. 45), the elongated plate member 5610 is replaced by a rod 5810.

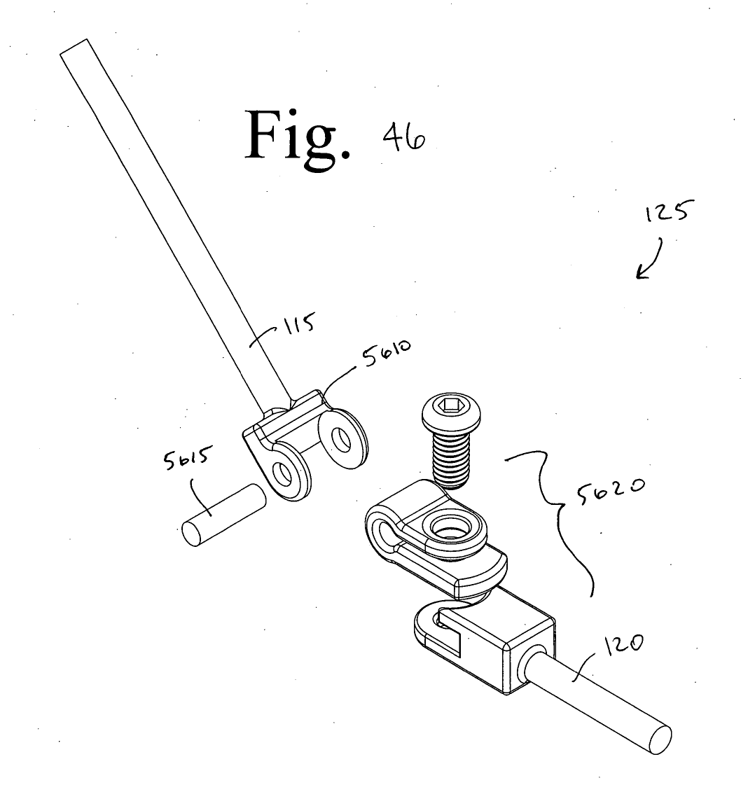

FIG. 46 shows an exploded view of an alternate embodiment of the connector. FIG. 47 shows an assembled view of the connector in FIG. 46. The device permits independent movement of the rods 115 and 120 in two non-parallel planes. The connector includes a single locking screw 5605 that can be used to tighten the device so as to fix the rods 115 and 120 in a desired position. The rod 115 includes a coupler 5610 on one end. The coupler 5610 is configured to rotate about a rotation pin 5615 along first axis R1. The second rod 120 includes a coupler assembly 5620 on one end. The coupler assembly 5620 is configured to rotate about a second axis R2.

FIGS. 48A and 48B show cross-sectional view of the connector of FIG. 47. Threaded locking screw 5605 interacts with complimentary threaded bore 5616 of assembly 5602. Locking screw 5605 is coupled to both the coupler 5610 and the coupler assembly 5620 such that, when locking screw 5605 is tightened, it compresses both coupler 5610 and coupler assembly 5620. When fully tightened, the locking screw 5605 exerts sufficient compression to prevent movement of the rods 115 and 120 relative to one another.

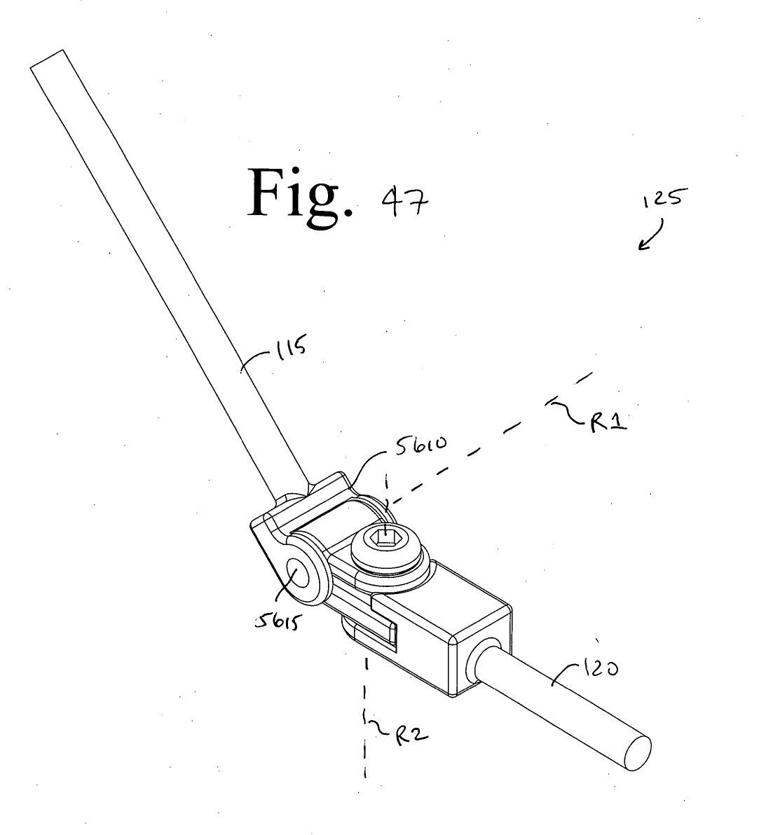

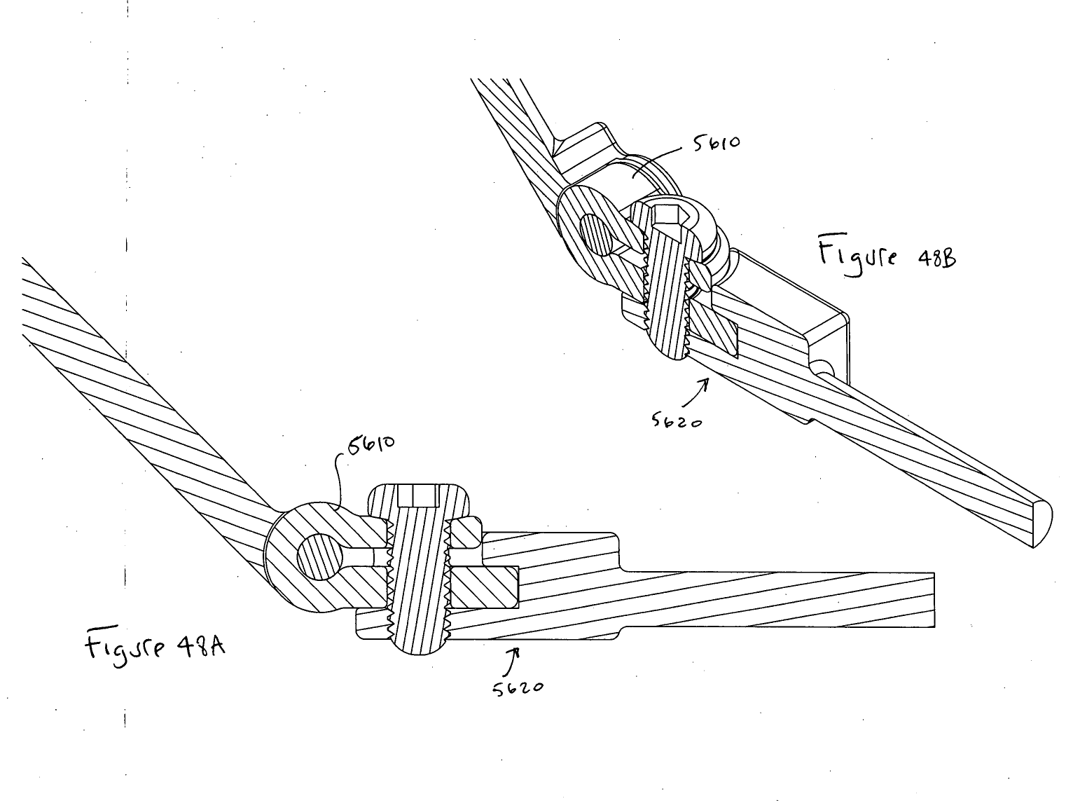

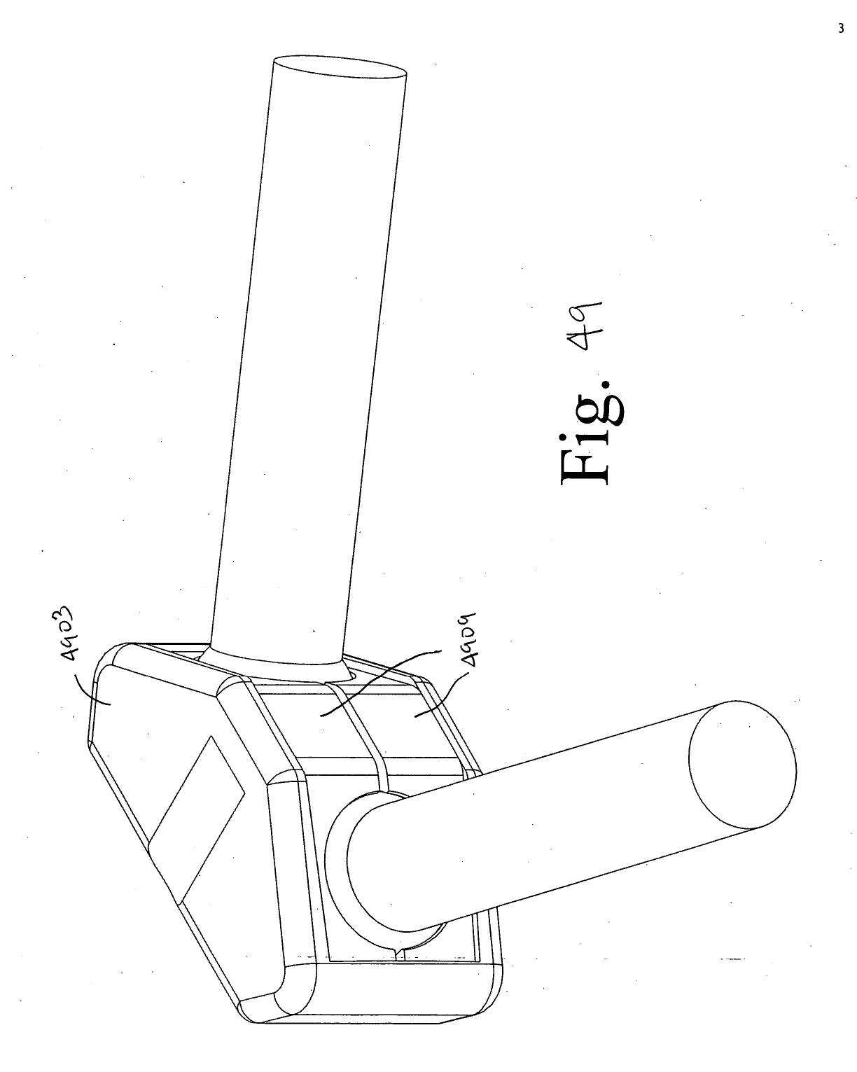

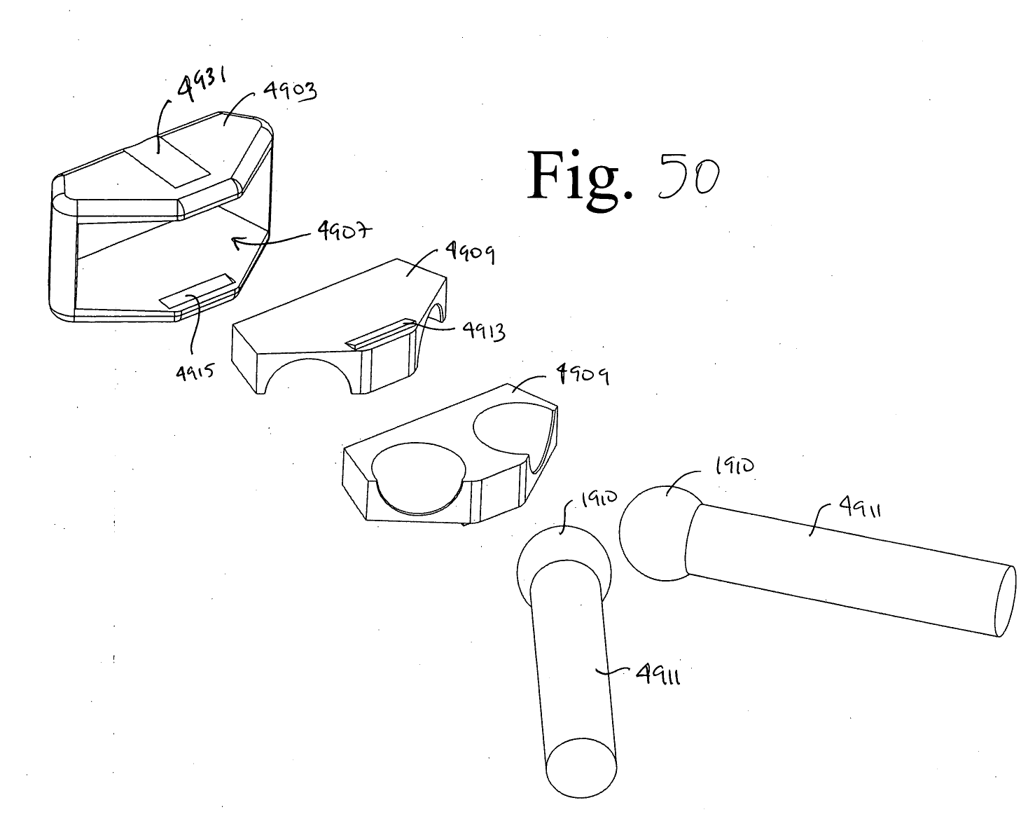

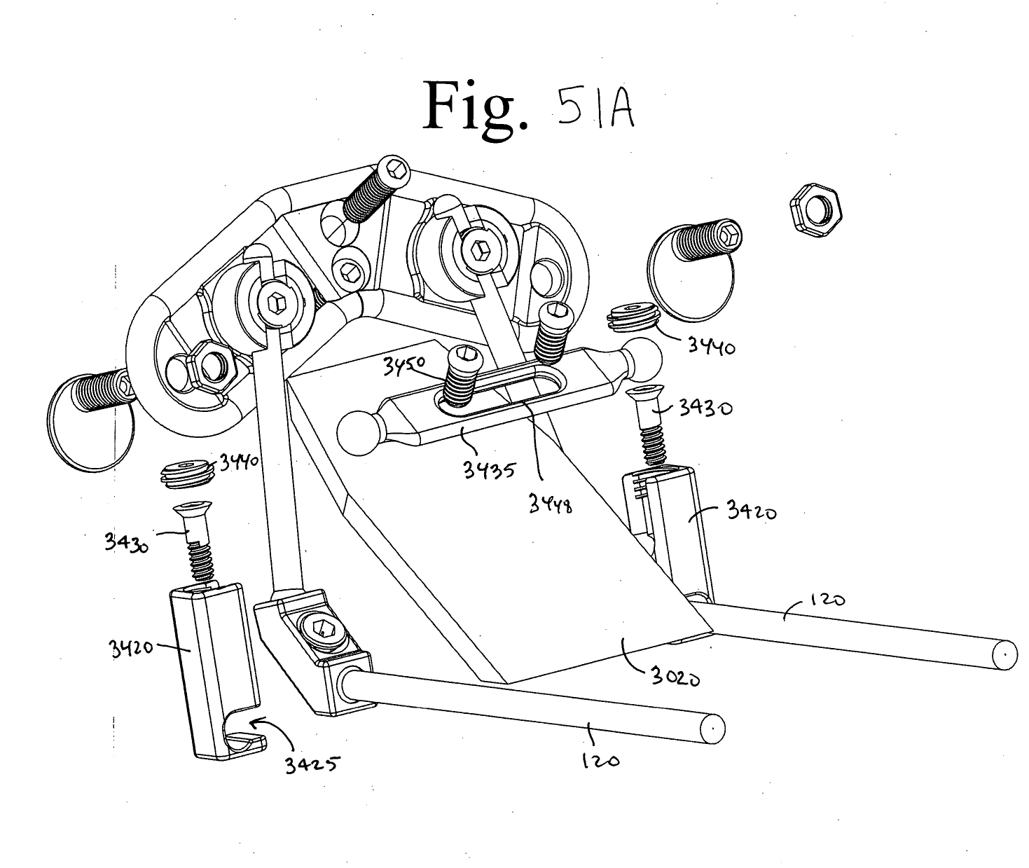

FIGS. 49 and 50 illustrate an additional connector embodiment. The connector includes an outer housing 4903 that has an internal cavity 4907 which receives a pair of inner housings 4909. The inner aspects of housings 4909 collectively form two cavities that receive the heads 1910 of rods 4911. The outer surface of housings 4909 is tapered on one or more sides (preferably those surfaces that lie parallel to the long axis of the connector, such as surface 4910) so as to form a complimentary interference taper, such as a Morse taper, with one or more inner surfaces of outer housing 4903 (such as surface 4904). Further, each of inner housings 4909 has a protrusion 4913 that mates with the corresponding indentation 4915 located on the inner surfaces of outer housings 4903. In use, rods 4911 are already seated within inner housings 4909 and loosely held within outer housing. Rods 4911 are placed into the desired position relative to one another. A locking instrument (not shown) attaches onto a cut-out 4931 located on each side of the outer surface of outer housing 4903. The instrument applies a force onto inner housings 4909 so as to compress them deeper into cavity 4907. The compressive force placed onto inner housings 4909 by the interaction of the tapered surfaces of the inner housings and tapered surface inside the outer housings will cause heads 1910 of rods 4911 to be immobilized within housing 4909.

Although an interference taper locking mechanism (such as a Morse taper) provides powerful immobilization, it may be loosened with only modest back-out of the inner housing members relative to the outer housing. This is prevented by the capture of protrusions 4903 within indentations 4915 of the outer housing. An alternative locker of the taper mechanism may be produced by using ratchets, wedge locker, locking rings and the like.

Graft Retainers

The occipito-cervical fixation system 100 is adapted to engage a bone graft 3020. In this regard, the system 100 can include a bone graft retainer and cross-link assembly 3010 that is configured to attach the bone graft to rods 120 and/or rods 115. Assembly 3010 serves to rigidly retain the bone graft at the desired site of fusion and to apply a compressive force onto the graft in order to maximize the likelihood of bony fusion. In addition, the assembly can function as a cross-link between inter-connecting rods and thereby increase the rigidly and rotational stability of the whole construct.

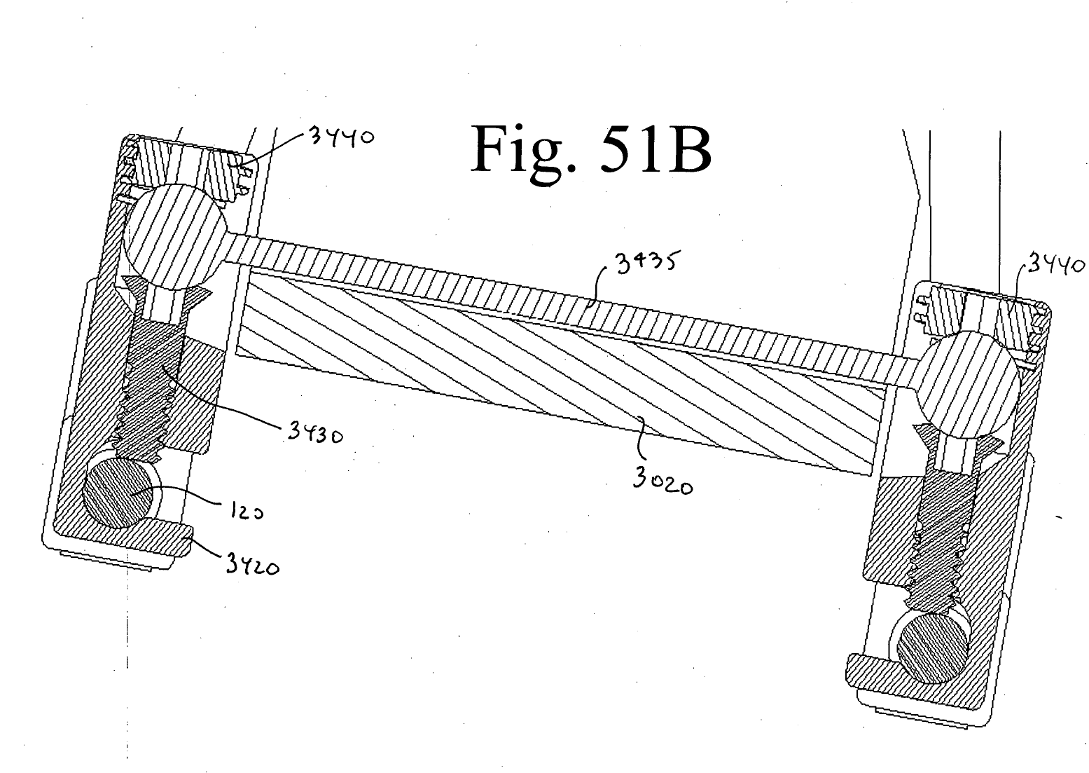

FIG. 51A shows an exploded view of one embodiment of a combination bone graft retainer and cross-link assembly 3010. The assembly includes a pair of rod attachments 3420 that have slots 3425 that are sized and shaped to receive the rods 115 and/or 120 therein. A tightening screw 3430 can be threaded into the central bore of rod attachments 3420 so as to tighten each rod attachment 3420 onto a respective rod. A cross-link 3435 extends between and connects to the rod attachments 3420 in one of several manners. In the illustrated embodiment, each end of the cross-link 3425 includes a sphere that can be inserted into the central bore of the rod attachments 3420. A lock nut 3440 can be used to attach and lock the cross-link to the rod attachments. The cross-link includes a slot 3448 that receives one or more graft attachment screws 3450. Screws 3450 include threaded portions that can be threaded into the graft 3020.

FIG. 51 B shows a cross-sectional view of the attached and locked graft retainer and cross-link assembly. As illustrated, the graft 3020 is rigidly attached to the occipito-cervical fixation system 100 and it is thereby retained at the site of desired fusion. The assembly also imparts a compressive load onto the graft so as to increase the likelihood of fusion. Lastly, it enhances construct stability by cross-linking two or more rods.

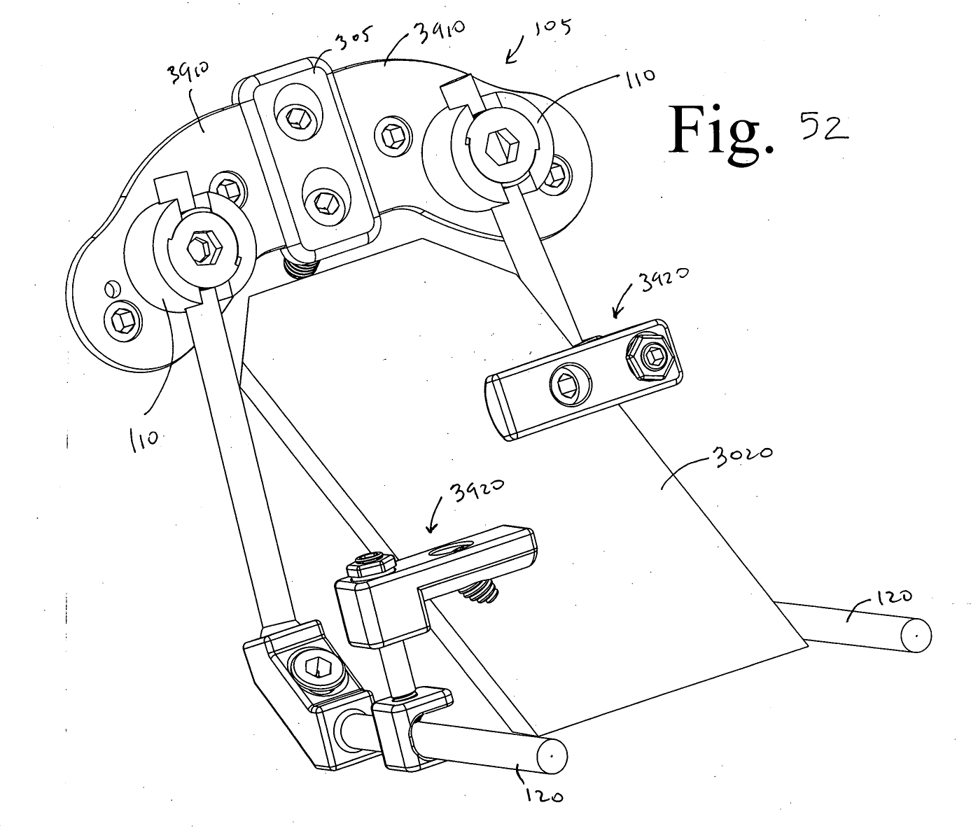

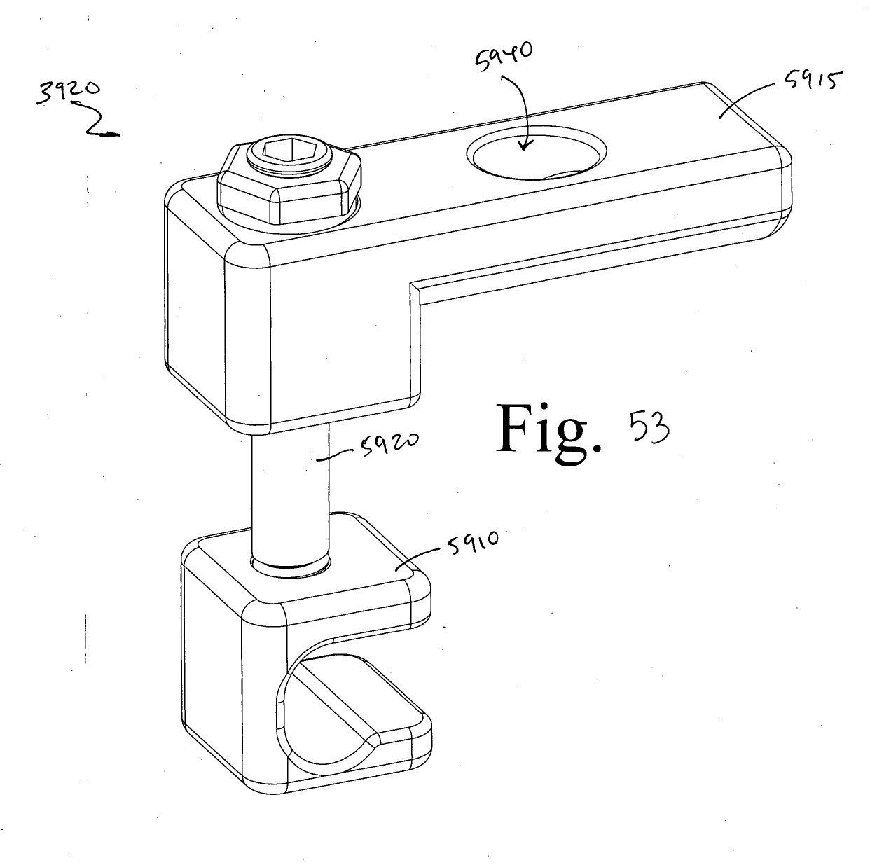

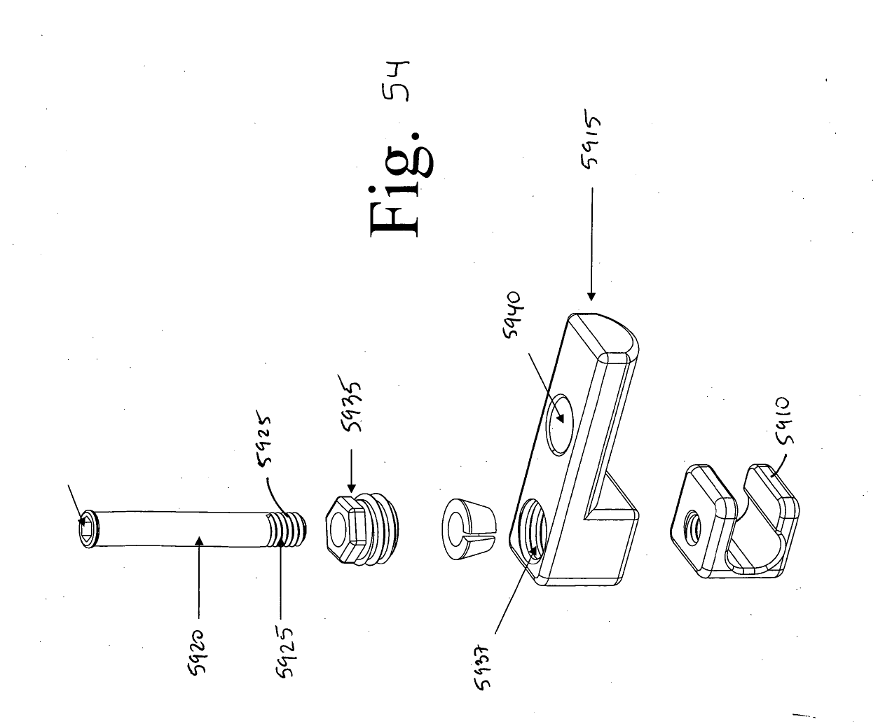

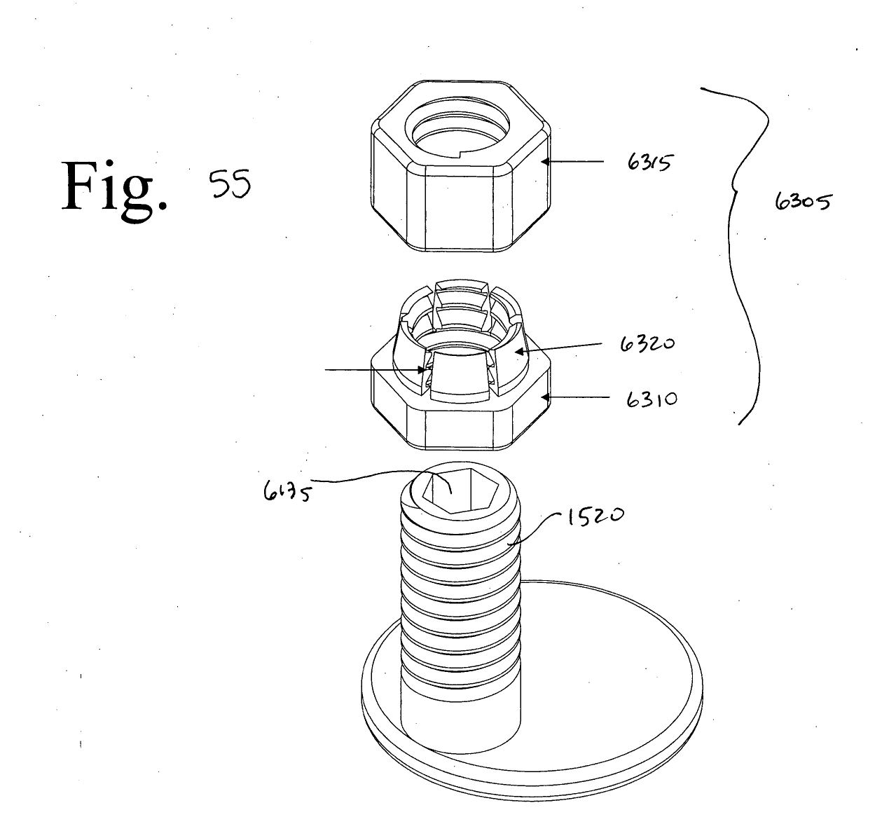

Another embodiment of a graft retainer assembly is shown in FIG. 52. In this embodiment, graft retainer 3920 serves to retain the graft apply a compressive load to it. The retainer 3920 does not extent between two rods and does not retain the cross-link feature. FIG. 53 shows a perspective view of the graft retainer 3920 while FIG. 54 shows an exploded view. Retainer 3920 includes a rod attachment member 5910 that attaches to a rod, and a graft attachment member 5915 that attaches to the graft 3020. An elongate extender 5920 connects the rod attachment member 5910 to the graft attachment member 5915. In this regard, the elongate extender 5920 includes a threaded end 5925 that mates with a hole in the upper end of the rod attachment member 5910. The elongate extender 5920 can be tightened to fixedly attach the rod attachment member 5910 to the rod 120, as shown in FIG. 53. The upper end of the elongate extender 5920 attaches to a lock nut 5935 for attaching the elongate extender 5920 to the graft attachment member 5915. The lock nut mates with a borehole 5937 in the graft attachment member 5915.

The graft attachment member 5915 includes a borehole 5940 that can receive a graft screw for attaching a graft to the graft attachment member 5915. Since bone healing is enhanced by compressive load, the graft attachment member 5915 can be used to compress the bone graft against the underlying native bone and promote bone healing. The undersurface of the graft attachment member 5815 can be curved or can have any contour so as to provide maximal bone contact regardless of the rotational angle along the long axis of the graft attachment member.

Button Fastener

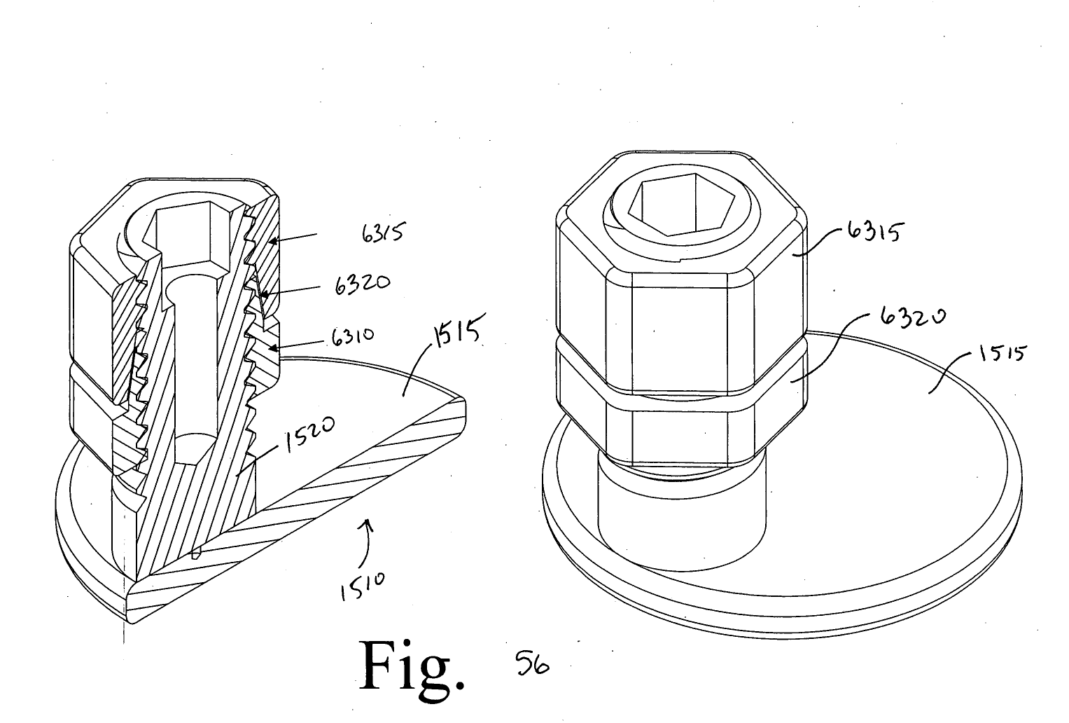

FIG. 55 shows an embodiment of a button attachment 1510 that includes a post 1520 that mates with a locking nut 6305. FIG. 56 shows views of the locking nut mounted onto the button attachment. The post 1520 includes a hex or other-shaped cavity 6135 for holding the post 1520 stationary while the nut 6130 is tightened. The locking nut 6305 is configured to reduce the likelihood of loosening from repetitive motion. The nut 6305 includes a first subunit 6310 and a second subunit 6315 positioned above the first subunit 6310. The first subunit 6310 includes an upwardly-extending stem 6320 that has one or more cuts 6320 that permit the stem 6320 to flex inward. The stem 6320 fits within an interior core of the second subunit 6315 when the two subunits are threaded onto the post 1520. As the second subunit 6315 is threaded downward over the first subunit 6310, an angled inner surface of the second subunit 6315 exerts a centripetal force onto the stem 6320 to cause the stem 6320 to constrict upon the post 1520. This creates a secure attachment between the nut 6305 and the post 1520. It should be appreciated that various designs of locking nuts can be used with the button attachment.

A number of embodiments have been described. Nevertheless, it will be understood that various modifications may be made without departing from the spirit and scope of the claims. Accordingly, other embodiments are within the scope of the following claims.

What is claimed is:

1. A skull attachment assembly for spinal fixation, comprising:
a skull attachment member adapted to be attached to a skull, the skull attachment member having a central member and first, second, and third arms extending outwardly from the central member, wherein the central body or the arms include at least one borehole adapted to receive a bone fastener for fastening the skull attachment member to a skull.

2. The skull attachment assembly of claim 1, further comprising at least one fixation assembly mounted on at least one of the arms, the fixation assembly configured to attach an elongate interconnector to the skull attachment member, wherein the fixation assembly can be fixed in place on at least one of the extensions.

3. The skull attachment assembly of claim 1, wherein the fixation assembly can move relative to the arm prior to being fixed in place.

4. The skull attachment assembly of claim 1, wherein at least a portion of the fixation assembly can rotate relative to the arm.

5. The skull attachment assembly of claim 1, wherein at least a portion of the fixation assembly can swivel relative to the arm.

6. The skull attachment assembly of claim 1, wherein the first and second arms extend outwardly from a lower portion of the central member and the third arm extends outwardly from an upper portion of the central member.

7. The skull attachment assembly of claim 6, wherein a fourth arm extends outwardly from an upper portion of the central member.

8. The skull attachment assembly of claim 1, further comprising an elongate interconnector comprised of an elongate rod or an elongate plate that attaches to the skull attachment member.

9. A skull attachment assembly, comprising:
a skull attachment member configured to be attached to a skull, the skull attachment member including a rod-based skeleton that supports at least one extension that provides a platform for attaching the skull attachment to a component.

10. The skull attachment assembly of claim 9, further comprising at least one rod fixation assembly mounted on at least one of the extensions, the rod fixation assembly configured to attach a rod to the skull attachment member, wherein the rod fixation assembly can be fixed in place on at least one of the extensions.

11. The skull attachment assembly of claim 9, further comprising an elongate interconnector comprised of an elongate plate that directly attaches to the skull attachment member.

12. The skull attachment assembly of claim 10, wherein the rod fixation assembly can move relative to the extension prior to being fixed in place.

13. The skull attachment assembly of claim 10, wherein at least a portion of the rod fixation assembly can rotate relative to the extension.

14. The skull attachment assembly of claim 10, wherein at least a portion of the rod fixation assembly can swivel relative to the extension.

15. The skull attachment assembly of claim 9, wherein the rod fixation assembly comprises an outer member and a pair of locking members positioned within the outer member and adapted to lock onto a rod while the outer member locks onto an upward protrusion on the extension, wherein the rod can be pressed downward into the locking member to immobilize the rod in the locking members and clamp the locking members onto the upward protrusion.

16. The skull attachment assembly of claim 9, wherein at least one extension receives a bone screw or a button for attaching the skull attachment to the skull.

17. The skull attachment assembly of claim 16, wherein at least one of the extensions is adapted to lock to a bone screw.

18. The skull attachment assembly of claim 9, further comprising a first bone screw that couples to the assembly, wherein the first bone screw couples to the assembly in a manner that permits the first bone screw to enter the skull at a non-perpendicular angle relative to a surface of the skull.

19. The skull attachment assembly of claim 18, further comprising a second bone screw that couples to the assembly, wherein the second bone screw couples to the assembly in a manner that permits the second bone screw to enter the skull at a perpendicular angle relative to a surface of the skull.

20. An assembly for retaining a bone graft, comprising:
a device that rigidly affixes a bone graft in a fusion orientation from a skull to a cervical spine.

21. The assembly of claim 20, wherein the device is adapted to attach to instrumentation that immobilizes the skull relative to the spine.

22. The assembly of claim 20, wherein the device is adapted to attach to a bone.

23. The assembly of claim 20, wherein the device has a first portion that attaches to a spinal rod and a second portion that attaches to a bone graft to secure the bone graft relative to the spinal rod.

24. The assembly of claim 23, wherein the attachment device attaches to a single spinal rod.

25. The assembly of claim 24, wherein the attachment device is adapted to extend between a pair of spinal rods so that the attachment device cross-links the spinal rods to one another.

26. A skull-spine connection assembly, comprising:
a connector adapted to connect at least two elongate spinal interconnectors to one another, the connector being adapted to transition between a locked state and an unlocked state, wherein the connector permits relative movement between the interconnectors throughout one or more planes when in the unlocked state, and wherein the connector immobilizes the interconnectors relative to one another when in the locked state.

27. The connection assembly of claim 26, wherein the connection assembly further comprises a skull interconnector device and a spine interconnector that are attached to the connector.

28. The connection assembly of claim 27, wherein the connector is integrally attached to a first interconnector and removably attached to a second interconnector.

29. The connection assembly of claim 27, wherein both connector device is integrally attached to a first interconnector and to a second connector.

30. The connection assembly of claim 27, wherein both connector device is removably attached to a first interconnector and to a second connector.

31. The connection device of claim 27, wherein the connector includes a locking member that is actuated to transition the connector between the locked and unlocked state.

32. The connection device of claim 27, wherein the connector comprises:
at least one housing connected to a first end of the skull interconnector and a first end of a spine interconnector; and
a locking member coupled to the housing, the locking member configured to move into a locked position wherein the locking member lockingly engages the first ends of the skull interconnector and the spine interconnector to immobilize the skull interconnector and the spine interconnector relative to one another;
an actuator configured to actuate the locking member into the locked position.

33. A skull attachment assembly, comprising:
a skull attachment device having a first borehole for receiving the shank of a bone screw;
a protrusion extending upward from the skull attachment device; and
at least one fixation assembly mounted on the protrusion, the fixation assembly configured to attach an elongate interconnector to the skull attachment device.

34. The skull attachment assembly of claim 33, wherein the fixation assembly comprises an outer member and a pair of locking members positioned within the outer member and adapted to lock onto a rod while the outer member locks onto the protrusion, wherein the rod can be pressed downward into the locking members to immobilize the rod in the locking members and clamp the locking members onto the protrusion.

35. The skull fixation assembly of claim 33, wherein the fixation assembly is movable relative to the skull attachment device prior to locking of the fixation assembly.
